(19) United States
(12) Patent Application Publication
(10) Pub. No.: US 2005/0193432 A1
(43) Pub. Date: Sep. 1, 2005

(54) Xenograft model of functional normal and malignant human breast tissues in rodents and methods thereof

(75) Inventors: Charlotte Kuperwasser, Boston, MA (US); Robert A. Weinberg, Cambridge, MA (US)

Correspondence Address: FISH & NEAVE IP GROUP;ROPES & GRAY LLP, ONE INTERNATIONAL PLACE, BOSTON, MA, 02110-2624, US

(73) Assignee: Whitehead Institute for Biomedical Research, Cambridge, MA (US)

(21) Appl. No.: 10/990,993

(22) Filed: Nov. 17, 2004

Related U.S. Application Data
(60) Provisional application No. 60/520,993, filed on Nov. 18, 2003.

(51) Int. Cl. A01K 67/027; C12N 5/06

(52) U.S. Cl. 800/18; 800/7; 435/354

(57) ABSTRACT

The invention relates to an orthotopic xenograft rodent model in which the stromal and epithelial components of the reconstructed mammary gland are of human origin, and to methods of making the mouse. The invention also relates to methods of using the rodent, including methods of identifying agents or drugs that inhibits abnormal growth of human breast epithelial cells or that inhibit the formation of breast tumors or other hyperplastic growths.

### CROSS-REFERENCE TO RELATED APPLICATIONS

This application claims the benefit of the filing date of U.S. Application No. 60/520,993, filed Nov. 18, 2003, entitled “XENOGRAFT MODEL OF FUNCTIONAL NORMAL AND MALIGNANT HUMAN BREAST TISSUES IN MICE AND METHODS THEREOF.” The entire teachings of the referenced application are incorporated by reference herein.

### FUNDING

Work described herein was funded, in whole or in part, by National Institutes of Health/NCI Grant Number CA80111-03 and by the Jane Coffin Child Foundation. The United States government has certain rights in the invention.

### BACKGROUND OF THE INVENTION

Cancer is considered to be a serious and pervasive disease. The National Cancer Institute has estimated that in the United States alone, 1 in 3 people will be afflicted with cancer during their lifetime. Moreover approximately 50% to 60% of people contracting cancer will eventually die from the disease. One particularly prevalent form of cancer, especially among women, is breast cancer. The incidence of breast cancer, a leading cause of death in women, has been gradually increasing in the United States over the last thirty years. In 1997, it was estimated that 181,000 new cases were reported in the U.S., and that 44,000 people would die of breast cancer (Parker et al, 1997, CA Cancer J. Clin. 47:5-27; Chu et al, 1996, J. Nat. Cancer Inst. 88:1571-1579).

The study of normal breast epithelial morphogenesis and carcinogenesis in vivo has largely utilized rodent models. Efforts at studying mammary morphogenesis and cancer with xenotransplanted human epithelial cells have failed to recapitulate the full extent of development seen in the human breast. It would be helpful to have an animal model which more closely mimics human breast tissue morphogenesis and carcinogenesis. Such animals may allow the identification of therapeutic agents to treat or prevent cancer, as well as in the identification of genes which contribute to or which antagonize tumorigenesis.

### SUMMARY OF THE INVENTION

The present invention relates to novel rodent models comprising allogenic grafts. One aspect of the invention relates to a mouse model, referred to as an orthotopic xenograft mouse model, in which the stromal and epithelial components of the reconstructed mammary gland are of human origin. The reconstructed human mammary glands form functional human breast ducts and lobules. Genetic modification of human stromal cells prior to implantation of ostensibly normal human mammary epithelial cells resulted in the outgrowth of benign and malignant lesions in the mice. This experimental model makes it possible to study human epithelial morphogenesis and differentiation in vivo under conditions which mimic those of the human breast. In addition, the model is useful to assess the effects of agents on breast tissue. For example, it can be used to identify or assess therapies, such as drugs, hormones, polypeptides, surgical techniques, radiation methods, or gene therapies for breast abnormalities, such as cancer, mastitis and fat necrosis. It is also useful to identify or assess factors or conditions, such as genetic makeup, environmental conditions, dietary patterns, stress, sexual behavior, bacterial or viral infections, immune responses, or exercise that may contribute to the development of breast abnormalities or confer protection against such abnormalities.

Furthermore, this experimental model is useful to identify genes that suppress or contribute to the development of breast abnormalities. This can be accomplished by altering gene activity in any of the components of the orthotopic xenograft mouse model described herein, including in the mouse adipose tissue, mouse fibroblasts, human fibroblasts or human epithelial cells, or in a combination thereof. Gene activity can be altered by numerous means, such as by introducing an exogenous gene or transgene, either wild-type or mutant, generating knockout mutations or gain-of-function mutations, or reducing gene activity through, for example, RNA interference or antibodies.

In a particular embodiment, the present invention is a mouse model in which the stromal component and the epithelial component of mammary glands are of human origin and the mammary glands form human breast ducts and/or lobules. In a specific embodiment, the human breast ducts are functional human breast ducts, such as human breast ducts which produce milk when the mouse is pregnant. In another specific embodiment, the mouse is an immunocompromised mouse in which the stromal component and the epithelial component of mammary glands are of human origin and the mammary glands form functional human breast ducts and lobules. Human breast tissue in the mouse model can be normal or abnormal. For example, it can mimic the characteristics of normal (nondiseased) human breast tissue or of abnormal human breast tissue, such as breast tissue with benign or malignant lesions. Normal and abnormal tissue can be obtained from women or men, using known techniques, such as those described herein. Alternatively, abnormal tissue can be produced by genetic modification of human stromal cells, human epithelial cells or both types of cells implanted into mice to produce a xenograft. Human stromal cells and/or human epithelial cells can be genetically modified prior to or after implantation into mice.

The invention also provides methods of generating the orthotopic xenograft mouse model. In one embodiment, the invention is a method of producing a humanized mammary fat pad, wherein human breast fibroblast cells are interspersed in mouse mammary adipose tissue, the method comprising: (a) generating nontumorigenic human mammary stromal fibroblasts; (b) treating the nontumorigenic human mammary stromal fibroblasts to induce their proliferation and their invasion into a mouse fat pad and introducing the nontumorigenic human mammary stromal fibroblasts into a cleared mammary fat pad from an immunocompromised mouse; and (c) allowing sufficient time for the nontumorigenic human mammary stromal fibroblasts to divide and invade into the cleared mammary fat pad, thereby producing a humanized mammary fat pad. In a specific embodiment, the nontumorigenic human mammary stromal fibroblasts comprise immortalized nontumorigenic human mammary stromal fibroblasts.

Furthermore, the invention provides a method of generating a mouse having at least one humanized mammary gland by introducing a composition comprising human mammary epithelial cells or human breast stem cells or both, into the humanized mammary fat pad of a mouse and allowing sufficient time for an epithelial outgrowth to develop, thereby forming a humanized mammary gland. U.S. patent Publication No. 2004/0037815 describes, in Example 2, methods for the isolation of breast stem cells, and WO03/060108 describes breast stem cells capable of differentiating into cells of mammary gland luminal epithelial and myoepithelial cell lineages and methods of isolating such cells. Additional publications describing the isolation of human breast stem cells include Clayton et al. Exp Cell Res. 2004;297(2):444-60; Boecker W et al., Cell Prolif. 2003;36 Suppl 1:73-84; and Welm et al. Cell Prolif. October 2003 ;36 Suppl 1:17-32.

In a specific embodiment, the humanized mammary gland comprises human breast ducts. In a further embodiment, the human breast ducts are functional and produce milk when the mouse is pregnant, when the mouse is treated with hormones which mimic the pregnant state, or both.

The invention further provides a modification of the mice and methods described herein in which the human mammary stromal fibroblast are replaced with non-human, non-mouse mammary stromal fibroblasts which are capable of supporting the morphogenesis of human mammary epithelial cells into human breast ducts. In a specific embodiment, the non-human, non-mouse mammary stromal fibroblasts comprise primate mammary stromal fibroblasts or mammary stromal fibroblasts from a species that is closely related evolutionarily to humans, such as a species from the same genus as humans.

Another embodiment of the invention relates to methods of screening for agents that modulate the growth, differentiation or morphogenesis of breast epithelial cells. In one aspect, the invention provides a method of determining whether an agent affects the growth, differentiation or morphogenesis of human breast epithelial cells, comprising (a) contacting the agent with a humanized mammary gland of a mouse, wherein the humanized mammary gland is comprised of (i) a humanized mammary fat pad comprised of human stromal fibroblast cells interspersed in mouse adipose tissue; (ii) an epithelial outgrowth comprised of human breast epithelial cells; and (iii) human breast; and (b) detecting an effect of the agent on the growth of the breast epithelial cells; and (c) selecting the agent which increases or decreases the growth of the breast epithelial cells.

In related aspects, the invention provides methods for identifying a drug that inhibits abnormal growth of human breast epithelial cells, comprising (a) contacting a candidate drug to be assessed with a humanized mammary gland of a mouse, wherein the humanized mammary gland is comprised of (i) a humanized mammary fat pad comprised of human stromal fibroblast cells interspersed in mouse adipose tissue; (ii) an epithelial outgrowth comprised of human breast epithelial cells; and (iii) human breast ducts; and (b) determining if the candidate drug inhibits abnormal growth of the human breast epithelial cells; and (c) selecting the candidate drug that inhibits abnormal growth of the human breast epithelial cells.

### BRIEF DESCRIPTIONS OF THE DRAWINGS

FIGS. 1A-1F show results of GFP of whole-mount and histological analysis of human-murine chimeric fat pads. FIGS. 1A and 1B are immunoflourescence wholemount micrographs showing GFP flourescence of cleared mouse mammary fat pads injected with immortalized human breast fibroblasts 4 weeks post injections (10×). Single GFP positive cells can be seen infiltrating the adipose stroma (B-inset). FIG. 1C is a micrograph of an hematoxylin and eosin (H&E)-stained section of an area of fibroblastic cord growth within the cleared fat pad. FIGS. 1D and 1E are serial sections of this region examined by immunohistochemistry using antibodies against human-specific vimentin (purple, FIG. 1D) or PCNA (brown, FIG. 1E). Genomic fluorescence in situ hybridization (FISH) was performed using specific probes for mouse Cot I gene (red or dark gray) and human genomic DNA (green or light gray), showing interpersed cells of both species (FIG. 1F).

FIG. 2A-2E show results of GFP of wholemount analysis of breast tissue of human breast and xenografted outgrowths. FIG. 2A is a section of normal human breast tissue isolated from reduction mammoplasty, stained with hematoxylin, and wholemounted which exhibits both ductal and TDLU (in brackets) structures. FIGS. 2B-E are carmine-stained wholemount sections of an outgrowth from xenografts in humanized fat pads which contains both ductal (FIG. 2B) and TDLU structures (FIGS. 2C,2D). Arrows point out commonly observed acini structures with hollow lumina. TDLU formation occurred during murine puberty (FIG. 2C) or pregnancy (FIG. 2D). Some outgrowth displayed evidence of both ductal and TDLU-like growths (Figure E).

FIGS. 3A-3B show results of GFP of epithelial and stromal cell contributions in xenograft breast tissue. Genomic FISH was performed on sections from mice in which human breast epithelial cells were injected into the humanized fat pads along with normal primary breast fibroblasts and a fluorescent micrograph is shown in FIG. 3A. Human cells (green or light gray) comprise all of the epithelial cells in the xenografts, but the stroma is comprised of both human and mouse cells (red or dark gray). A significant amount of angiogenesis is observed within the xenograft stromal regions shown in the stained section of micrograph FIG. 3B. H&E stained section reveals many capillaries (arrows) within the stroma indicative of neo-vascularization.

FIGS. 4A-4H show results of GFP of histological and molecular analysis of xenograft breast tissues. FIGS. 4A and 4B are paraffin sections of breast xenograft ductal outgrowths stained with hematoxylin and eosin. FIG. 4C is a double-labeled section of breast marker ductal structures; myoepithelial cell-specific expression of smooth-muscle actin is not detected in cells expressing the estrogen receptor. FIG. 4D is an immunohistochemistry-stained section with antibodies specific for PCNA, which demonstrates the proliferative state of the outgrowth. Brown nuclei can be detected in both epithelial and stromal cells. Lumenal-specific expression of cytokeratin 19 is detected in ER expressing cells in the section shown in FIG. 4E, while E-cadherin expression is detected on the membrane of luminal epithelial cells of the section shown in FIG. 5F. Paraffin sections of fully differentiated breast xenograft ductal outgrowths in pregnant mice were stained with hematoxylin and eosin in the section shown in FIG. 5G. Milk protein and fat droplets are seen (arrows) in the differentiated epithelial cells under the control of the hormones of pregnancy. Immunohistochemistry was performed on these sections with antibodies against human-specific β-casein milk protein and are shown in the micrograph shown in FIG. 4H. Beta-casein expression is detected both in secretory epithelial cells as well as in the luminal space of the alveoli.

FIG. 5A-5F show results of GFP of hyperplastic and tumorigenic outgrowths derived from reduction mammoplasty epithelium. FIGS. 5A and 5B are H&E stained sections of hyperplasias that developed from normal organoids in the absence of normal admixed fibroblasts. FIG. 5C is a graph showing the frequency of tumor formation when reduction mammoplasty organoids were introduced directly into humanized stroma overexpressing TGFβ or HGF (O) or when the organoids were co-injected in normal primary human breast fibroblasts (O+F). Histology of the tumors arising in glands humanized with growth factor expressing stroma: stroma that overexpresses either HGF, shown in the micrograph of a section of FIG. 5D or TGFβ1 shown in the micrograph of a section of FIG. 5E. Histology of outgrowths of various presumed stages of human breast cancer detected in the same field are shown in the micrographed section of FIG. 5F; normal ductal tissue (normal), benign hyperplastic ducts (hyperplasia), in-situ ductal cancer (DCIS) and invasive carcinoma (invasive).

### DETAILED DESCRIPTION OF THE INVENTION

### I. OVERVIEW

Described herein are rodent models comprising functional human breast tissues, methods of producing such rodent models and methods in which the rodents are used. The rodents of the present invention are useful to better understand the complex heterotypic mechanisms of normal human breast development and tumorigenesis, to identify therapies, such as drugs, for the treatment and/or inhibition of development/progression of breast lesions, which can be benign, precancerous or cancerous (malignant), and to identify genes which may serve as drug targets.

The invention described herein provides rodents having a humanized mammary fat pad which supports the growth and/or morphogenesis of human mammary epithelial cells into a outgrowth comprising human breast ducts, and rodents having a humanized mammary gland comprised of human mammary epithelial cells. The invention also provides methods of making the rodents described herein. Furthermore, the invention provides methods of using the rodents described in the invention, including methods of screening for agents, such as drugs, that inhibit or enhance the abnormal growth and/or morphogenesis of humanized mammary glands.

Although many of the methods described herein are generally described with reference to mice, they are generally applicable to any rodent. Preferred rodents include members of the Muridae family, which includes rats and mice. In one embodiment, the rodent is selected from rabbits, rats, hamsters, and mice. Similarly, although the orthotopic xenograft models are described with particular reference to the mouse, the invention further provides similar models based on other rodents, such as rabbits, rats and hamsters.

One aspect of the invention provides a rodent, such as a mouse, wherein at least one mammary fat pad is a humanized mammary fat pad which comprises nontumorigenic xenogenic mammary stromal fibroblasts interspersed in mouse adipose tissue and supports the morphogenesis of human mammary epithelial cells into human breast ducts. In one specific embodiment, the humanized mammary fat pad supports the growth and/or morphogenesis of (i) nontumorigenic human mammary epithelial cells; (ii) human mammary epithelial cells that are not immortal; (iii) primary human mammary epithelial cells; (iv) human mammary epithelial cells that do not express a recombinant gene; or (v) combinations thereof.

One aspect of the invention provides a rodent, such as a mouse, having at least one functional humanized mammary gland, wherein the humanized mammary gland comprises: (i) a humanized mammary fat pad comprised of nontumorigenic mammary stromal fibroblasts interspersed in mouse adipose tissue; and (ii) an epithelial outgrowth comprised of human breast epithelial cells. In one embodiment, the epithelial outgrowth comprises human breast ducts, such as human breast ducts that produce milk when the mouse is pregnant.

Another aspect of the invention provides a method of producing a humanized mammary fat pad in a mouse, or any other rodent, wherein the humanized mammary fat pad comprises human breast fibroblast cells interspersed in mouse mammary adipose tissue, the method comprising: (a) generating or providing nontumorigenic human mammary stromal fibroblasts; (b) inducing the proliferation and invasiveness of the nontumorigenic human mammary stromal fibroblasts and introducing the nontumorigenic human mammary stromal fibroblasts into a cleared mammary fat pad from the mouse; and (c) allowing sufficient time for the nontumorigenic human mammary stromal fibroblasts to divide and invade into the cleared mammary fat pad, thereby producing a humanized mammary fat pad.

Another aspect of the invention provides a rodent, such as a mouse, which comprises at least one humanized mammary fat pad generated according to any of the methods described herein. Similarly, yet another aspect of the invention provides a rodent, such as a mouse, which comprises at least one humanized mammary gland generated according to any of the methods described herein.

The invention further provides a method of generating a humanized mammary gland, comprising (a) generating a humanized mammary fat pad; (b) introducing a composition comprising (1) human mammary epithelial cells; or (2) human breast stem cells; or (3) a combinations thereof, into the humanized mammary fat pad; and (c) allowing sufficient time and appropriate conditions for an epithelial outgrowth to develop, thereby forming a humanized mammary gland. In one embodiment, the composition does not comprise tumorigenic cells. In another embodiment, the composition comprises less than 50%, 40%, 30%, 25%, 20%, 15%, 12%, 10%, 8%, 5%, 4%, 3%, 2%, 1%, 0.5%, 0.2%, 0.1% or 0.01% tumorigenic cells. In another embodiment, the composition comprises primary tumorigenic cells. In another specific embodiment, the composition comprises human mammary epithelial cells. In another embodiment, the human mammary epithelial cells (i) are nontumorigenic human mammary epithelial cells, (ii) are not genetically engineered; (iii) do not express a recombinant transgene; or (iv) combinations thereof.

The invention also provides methods of using the rodents. One aspect of the invention provides a method of identifying an agent that affects the growth of human breast epithelial cells, comprising (a) contacting a humanized mammary gland of a mouse with the agent, wherein the humanized mammary gland is comprised of (i) a humanized mammary fat pad comprised of human stromal fibroblast cells interspersed in mouse adipose tissue; and (ii) an epithelial outgrowth comprised of human breast epithelial cells; and (iii) human breast ducts; and (b) detecting an effect of the agent on the growth of the breast epithelial cells; and (c) selecting the agents which increases or decreases the growth of the breast epithelial cells.

Similarly, the invention provides a method of identifying a drug that inhibits abnormal growth of human breast epithelial cells, comprising (a) contacting the humanized mammary gland of a mouse with the drug, wherein the humanized mammary gland is comprised of (i) a humanized mammary fat pad comprised of human stromal fibroblast cells interspersed in mouse adipose tissue; (ii) an epithelial outgrowth comprised of human breast epithelial cells; and(iii) human breast ducts; (b) determining if the candidate drug inhibits abnormal growth of the human breast epithelial cells; and (c) selecting the candidate drug that inhibits abnormal growth of the human breast epithelial cells. The agent or drug may be adminstered through any suitable means. When the agent comprises a compound, the agent may be administered orally or by injection, such as by subcutaneous, intraperitoneally, intravenous or intraarterial injection the agent or drug may also be administered using an implantable device or any sustained or timed-release device. The agent or drug may also be adminstered systemically or locally.

### II. DEFINITIONS

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

The terms “polypeptide” and “protein” are used interchangeably herein.

The term “recombinant” is used herein to mean any nucleic acid comprising sequences which are not adjacent in nature. A recombinant nucleic acid may be generated in vitro, for example by using the methods of molecular biology, or in vivo, for example by insertion of a nucleic acid at a novel chromosomal location by homologous or non-homologous recombination.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term antibody also includes polyclonal, monoclonal, or other purified preparations of antibodies and recombinant antibodies.

The term “antineoplastic agent” is used herein to refer to agents that have the functional property of inhibiting a development or progression of a neoplasm or neoplastic cell growth in a human, particularly a malignant (cancerous) lesion, such as a carcinoma, sarcoma, lymphoma, or leukemia.

The term “sensitive to a drug” or “resistant to a drug” is used herein to refer to the response of a cell when contacted with an agent. A cancer cell is said to be sensitive to a drug when the drug inhibits the cell growth or proliferation of the cell to a greater degree than is expected for an appropriate control, such as an average of other cancer cells that have been matched by suitable criteria, including but not limited to, tissue type, doubling rate or metastatic potential. In some embodiments, greater degree refers to at least 10%, 20%, 30%, 40%, 50%, 60%, 70%, 80%, 90%, 100%, 200%, or 500%. A cancer cell is said to be sensitive to a drug when the drug inhibits the cell growth or proliferation of the cell to a lesser degree than is expected for an appropriate control, such as an average of other cancer cells that have been matched by suitable criteria, including but not limited to, tissue type, doubling rate or metastatic potential. In some embodiments, lesser degree refers to at least 10%, 15%, 20%, 25%, 50% or 100% less.

### III. RODENTS HAVING HUMANIZED MAMMARY FAT PADS AND METHODS OF MAKING SAME

The invention provides a method of producing a humanized mammary fat pad, in which nontumorigenic human mammary stromal fibroblasts cells are interspersed in rodent mammary adipose tissue. The method comprises (a) generating nontumorigenic human mammary stromal fibroblasts; (b) treating the nontumorigenic human mammary stromal fibroblasts to induce their proliferation and their invasion into a mouse fat pad and introducing the nontumorigenic human mammary stromal fibroblasts into a cleared mammary fat pad from a rodent; and (c) allowing sufficient time for the nontumorigenic human mammary stromal fibroblasts to divide and invade into the cleared mammary fat pad, thereby producing a humanized mammary fat pad. In a specific embodiment, the nontumorigenic human mammary stromal fibroblasts comprise immortalized nontumorigenic human mammary stromal fibroblasts. In a preferred embodiment, the rodent is a mouse

In one embodiment, from about two weeks to about four weeks are allowed for the nontumorigenic human mammary stromal fibroblasts to divide and invade into the cleared mammary fat pad. In another embodiment, at least one week is allowed.

The rodents, and mice in particular, may be grown in conventional ways. Depending upon the degree of immunocompromised status of the mouse, the mouse may be protected to varying degrees from infection. Thus, in some instances, a sterile environment or prophylactic antibiosis may be indicated. Prophylactic antibiosis may be achieved for SCID mice with 25-75 mg trimethoprim and 100-300 mg sulfamethoxazole in 5 ml of suspension or in 5 gm food pellets, given 3 days each week. Alternatively, it may be satisfactory to isolate the potential xenogeneic mice from other animals in germ-free environments after caesarean derivation. The feeding and maintenance of the mouse will for the most part follow conventional techniques.

Accordingly, the present invention also provides a mouse having at least one humanized mammary fat pad, wherein the humanized mammary fat pad comprises nontumorigenic human mammary stromal fibroblasts interspersed in mouse adipose tissue, and wherein the humanized mammary fat pad supports the morphogenesis of differentiation of human mammary epithelial cells into human breast ducts. In a specific embodiment, the human breast ducts comprise ducts and acini which produce milk.

In one embodiment, the mice of the present invention are immunocompromised mice. In a specific embodiment, the mice of the present invention are NOD/SCID mice. In other embodiments, the mice are nude mice, mice in which the thymus gland has been surgically removed, or mice in which the immune system has been suppressed by drugs or by genetic manipulations. In another embodiment, the mice of the methods described herein, or the mice provided by the invention, are RAG mice used. One skilled in the art may choose any strain of mice whose immune system is unable to reject the transplanted human cells.

The invention further provides a modification of the mice and the methods described herein in which the human mammary stromal fibroblast are replaced with non-human, non-mouse mammary stromal fibroblasts, herein referred to as non-human xenogenic mammary stromal fibroblasts, which are capable of supporting the morphogenesis of human mammary epithelial cells into human breast ducts, and in particular embodiments, into human breast ducts which are able to secrete milk when the mouse is pregnant. In a specific embodiment, the non-human, xenogenic mammary stromal fibroblasts comprise primate mammary stromal fibroblasts or mammary stromal fibroblasts from a specie that is evolutionarily closely related to humans. Non-human xenogenic mammary stromal fibroblast may be isolated from animals and manipulated using the methods described herein for the manipulation of human mammary stromal fibroblasts. For example the methods described herein for the immortalization and physiological activation of human mammary breast cells may also be applied to the non-human xenogenic mammary stromal fibroblast. In some embodiments, the non-human xenogenic mammary stromal fibroblast are isolated from primates. When non-human xenogenic mammary stromal fibroblasts are used in place of human mammary stromal fibroblasts, the resulting mammary fat pads are still referred to as humanized fat pads because they can support the proliferation and morphogenesis of human breast epithelial cells. In additional embodiments, non-human xenogenic mammary stromal fibroblasts may be used in combination with human mammary stromal fibroblasts, to generate humanized mammary fat pads.

The present invention provides mice which have at least one humanized mammary fat pad or at least one humanized mammary gland. In a specific embodiment, the #4 inguinal mammary gland of the mouse is modified to generate a humanized mammary fat pad. The generation of cleared mammary fat pads is described in U.S. Pat. No. 5,434,341 and in Outzen and Custer, J. Natl. Cancer Inst. (1975) 55:1461-1466, hereby incorporated by reference in their entirety. In another embodiment of the methods described herein, the mice contain two humanized mammary fat pads. In a particular embodiment, both the left and the right #4 inguinal mammary glands of the mouse are modified to generate two humanized mammary fat pads. When a mouse has two or more humanized mammary fat pads, each mammary fat pad may be identically or it may differ in at least one aspect. For example, one humanized mammary pad may be generated with human mammary stromal fibroblast that have been genetically modified to secrete a growth factors while the other humanized mammary fat pad contains human mammary stromal fibroblast that have not been genetically modified, or which secrete a different growth factor.

In one embodiment of the methods described herein, the nontumorigenic human mammary stromal fibroblasts are immortalized. As used herein, the term “immortalized nontumorigenic fibroblast” refers to fibroblasts which can divide repeatedly in cell culture without entering senescence, and which do not form tumors on nude mice or form colonies on soft agar. Immortalized nontumorigenic fibroblast can be generated by isolating human stromal fibroblasts from reduction mammoplasty tissue, from men or women, and expanding them in vitro, such as through the use of standard cell culture techniques. The primary human stromal fibroblasts are then immortalized by the ectopic expression of the catalytic subunit of telomerase or other proteins known to immortalize fibroblasts, such as the SV40 large T antigen. Combinations of proteins may also be used to improve the efficiency of immortalization, such as the SV40 large T antigen and c-myc. The telomerase catalytic subunit used can be human, mouse or other origin and can be exogenous DNA introduced into cells or endogenous DNA encoding the catalytic subunit. For example, ectopic expression of telomerase catalytic subunit can be achieved by turning on or upregulating the activity of an endogenous hTERT gene, such as by increasing activity of the hTERT promoter. Alternatively, a DNA vector encoding the catalytic subunit of telomerase, driven by a promoter which is transcriptionally active in human stromal fibroblasts, such as a viral promoter, is introduced as a transgene into the human stromal fibroblasts, to produce human stromal fibroblasts which ectopically express a catalytic telomerase subunit.

Methods for introducing transgenes into human fibroblasts are well-known in the art. In one embodiment, a plasmid encoding the human TERT subunit or the SV40 large-T antigen is introduced into primary human stromal fibroblast cells using a virus, such as a retrovirus or a lentivirus. Other methods can be used, such as electroporation or liposome-mediated transfection of vectors comprising DNA encoding human telomerase catalytic subunit. In a further embodiment, a GFP-encoding transgene is also introduced into the stromal fibroblast cells to facilitate their subsequent identification within the humanized mammary fat pad. Vectors for expressing the proteins described herein may be selected from commercial sources or constructed for a particular expression system. Such vectors may contain appropriate regulatory sequences, such as promoter sequences, terminator sequences, polyadenylation sequences, enhancer sequences and marker genes. Vectors may be plasmids, or viral-based.

In another embodiment, the human nontumorigenic mammary stromal fibroblasts used to generate the humanized mammary fat pad are genetically modified. In a specific embodiment, the human nontumorigenic mammary stromal fibroblasts are genetically modified to overexpress or ectopically express proteins, preferably secreted proteins, such as growth factors. Growth factors that can be used include transforming growth factor-β (TGF-β), hepatocyte growth factor (HGF), fibroblast growth factors such as FGF-7 and FGF-1, insulin-like growth factor (IGF), epidermal growth factor (EGF), colony stimulating factor 1 (CSF-1), platelet derived growth factor (PDGF), SDF-1 (CXCL12), and heregulin. In one embodiment, the nontumorigenic mammary stromal fibroblast cells are genetically modified to express at least one polypeptide, such as a human or a mouse polypeptide, wherein the polypeptide is selected from the group consisting of a chemokine, a cytokine, a growth factor, a tumor antigen or an antibody. Growth factors include Flt3L polypeptides, while tumor antigens include HER2/neu, CA15.3, CD31, CD105, Tie-2/Tek, NY-ESO-1, MTA1, MUC1, (CEA), Ep-CAM, p53, MAGE 1, 2, 3, 4, 6 or 12, and Thompson-Friedenreich antigen. Cytokines include but are not limited to IFN-α, IL-2, IL-4, IL-12 and GM-CSF. In other embodiments, the antibody comprises a monoclonal antibody, a humanized antibody, a single chain antibody or a chimeric antibody. In a preferred embodiment, the antibody is specific for a breast cancer antigen. In another embodiment, the antibodies is Rituxan, IDEC-C2B8, anti-CD20 Mab, Panorex, 3622W94, anti-EGP40 (17-1A) pancarcinoma antigen on adenocarcinomas Herceptin, Erbitux, anti-Her2, Anti-EGFr, BEC2, anti-idiotypic-GD₃ epitope, Ovarex, B43.13, anti-idiotypic CA125, 4B5, Anti-VEGF, RhuMAb, MDX-210, anti-HER2, MDX-22, MDX-220, MDX-447, MDX-260, anti-GD-2, Quadramet, CYT-424, IDEC-Y2B8, Oncolym, Lym-1, SMART M195, ATRAGEN, LDP-03, anti-CAMPATH, ior t6, anti CD6, MDX-11, OV103, Zenapax, Anti-Tac, anti-IL-2 receptor, MELIMMUNE-2, MELIMMUNE-1, CEACIDE, Pretarget, NovoMAb-G2, TNT, anti-histone, Gliomab-H, GNI-250, EMD-72000, LymphoCide, CMA 676, Monopharm-C, anti-FLK-2, SMART 1D10, SMART ABL 364, ImmuRAIT-CEA, or combinations thereof.

Nucleic acids (DNA, RNA) encoding growth factors can be introduced into vectors that drive their expression in fibroblast cells, and can be introduced into stromal fibroblast cells using retroviral vectors and other standard methods. Stromal fibroblasts ectopically expressing growth factors which promote the growth of human mammary epithelial cells are particularly desirable to induce abnormal cell growth and to increase the incidence of tumors in humanized mammary glands. Such mice can be used as a sensitized assay system to identity agents, such as drugs, that reduce the incidence or growth of mammary gland tumors. Thus, one aspect of the invention provides mice in which the human nontumorigenic stromal fibroblasts contained in their humanized mammary fat pad are genetically-modified to ectopically express secreted proteins such as growth factors.

In another embodiment, the mouse is genetically-modified to reduce expression of an endogenous gene, to overexpress an endogenous gene, or to ectopically express a gene, such as a human gene. In a specific embodiment, the mouse is a transgenic mouse which expresses a human growth factor. The transgene can be expressed in any desired tissue or cell type, such as fibroblasts, epithelial cells or adipocytes. In one embodiment, the transgene is expressed in breast tissue. In another embodiment, the transgene is a secreted polypeptide that can contact mammary epithelial cells. In another embodiment, the mice of the present invention are knockout mice in which the function of a gene of interest has been altered.

The human nontumorigenic stromal fibroblasts of the present invention are treated to induce their proliferation and invasion of the mouse mammary fat pad. In one embodiment, the fibroblasts are treated to induce a state of physiological activation. “Physiological activation” refers to a state in which the fibroblasts are induced to express proteases, matrix proteins such as collagen, growth factors, chemokines, smooth muscle actin and TGF-β that are characteristic of dividing fibroblasts and of fibroblast found at the sites of wound healing and inflammation. In one embodiment, the physiological activation comprises irradiating mammary stromal fibroblasts and introducing them together with nonirradiated human stromal fibroblasts into the cleared mammary fat pad of a mouse. In a particular embodiment, the irradiated stromal fibroblasts are also nontumorigenic human mammary stromal fibroblasts. In one embodiment, mammary stromal fibroblasts are irradiated with from about 0.2 to about 40 Gy of ionizing radiation, such as gamma radiation, e.g. from about 1 to about 10 Gy or from about 3 to 5 about Gy, and combined with non-irradiated nontumorigenic human mammary stromal fibroblasts. The ratio of irradiated to nonirradiated fibroblasts can be determined empirically. For example, the ratio can be from about 10 to 1 and about 1 to 10, such as from about 3 to 1 to about 1 to 3 or approximately 1 to 1. In another embodiment, all the nontumorigenic human mammary stromal fibroblasts to be injected into the fat pad are irradiated. In one embodiment, either the radiated mammary stromal fibroblasts or the non-irradiated mammary stromal fibroblasts or both are immortalized nontumorigenic human mammary stromal fibroblasts.

In still another embodiment, treatment of the mammary stromal fibroblasts to induce their proliferation and invasion into the mouse mammary fat pad comprises incubating the fibroblasts with conditioned media from fibroblasts which are themselves physiologically activated. In a specific embodiment, treatment of the mammary stromal fibroblasts to induce their proliferation and invasion into the mouse mammary fat pad comprises introducing one or more transgenes into the fibroblasts which induce their proliferation and tissue invasiveness, such as transgenes encoding growth factors or proteases.

To generate a humanized mammary fat pad, the composition containing nontumorigenic human stromal fibroblasts is injected into a cleared mouse mammary fat pad. A cleared mouse mammary fat pad is a fat pad in which the endogenous epithelium has been removed. The generation of cleared mammary fat pads is well-known to the artisan skilled in the art, and is described in DeOme et al, Cancer Res. 1959; 19:515-525, hereby incorporated by reference in its entirety. In one embodiment, the mammary fat pads are cleared when the mice are from about one week to about seven weeks old, from about two weeks to about four weeks old, or at about three weeks of age. After introducing the nontumorigenic human stromal fibroblasts into the cleared fat pad, sufficient time is allowed for the nontumorigenic human mammary stromal fibroblasts to divide and invade into the cleared mammary fat pad. In one embodiment, sufficient time is at least two weeks.

In some embodiments of the methods described herein to generate a humanized mammary fat pad, the nontumorigenic human mammary stromal fibroblasts are treated to induce their proliferation and their invasion into a mouse fat pad before they are introduced into the cleared mammary fat pad. In other embodiments, the nontumorigenic human mammary stromal fibroblasts are treated to induce their proliferation and their invasion into a mouse fat pad after they are introduced into the cleared mammary fat pad. When the treatment of the nontumorigenic human mammary stromal fibroblasts is performed after their introduction into the cleared fat pad, the treatment may comprise a localized or a systemic treatment. For instance, if radiation is used to activate the fibroblasts, the radiation can be administered locally to the area of the mammary fat pad, or the entire mouse can be irradiated. When radiation is administered locally, it may administered through a source of external radiation or through the temporary or permanent implantation of a radioactive composition.

### IV. RODENTS HAVING HUMANIZED MAMMARY GLANDS AND METHODS OF MAKING SAME

The invention provides methods of generating a humanized mammary gland, comprising (a) generating a humanized mammary fat pad according to any one of the methods described herein; (b) introducing a composition comprising human mammary epithelial cells into the humanized mammary fat pad; and (c) allowing sufficient time and appropriate conditions for an epithelial outgrowth to develop, thereby forming a humanized mammary gland. In some embodiments, of the methods described herein, the epithelial outgrowth comprises human breast ducts. In further embodiments, the human breast ducts make milk with the mouse is pregnant, when the mouse is hormonally stimulated, or when the humanized mammary gland is stimulated physically, or combinations thereof.

In alternate embodiments of the methods described herein, human breast epithelial stem cells, also known as human breast epithelial progenitor cells, are substituted in place of the human mammary epithelial cells, while in other embodiments, a composition comprising a combination of human mammary epithelial cells and human breast epithelial progenitor cells are introduced into the humanized mammary fat pad to generate a humanized mammary gland. Accordingly, the invention also provides a method of generating a humanized mammary gland, comprising (a) generating a humanized mammary fat pad according to any of the methods described herein; (b) introducing a composition comprising (1) human mammary epithelial cells; or (2) human breast stem cells; or (3) a combination thereof, into the humanized mammary fat pad; and (c) allowing sufficient time and appropriate conditions for an epithelial outgrowth to develop, thereby forming a humanized mammary gland.

The invention also provides a mouse generated according any of the methods provided by the invention. In one embodiment, the invention provides a mouse having at least one humanized mammary gland. In another embodiment, the invention provides a mouse having at least one humanized mammary gland, wherein the humanized mammary gland comprises (i) a humanized mammary fat pad comprised of nontumorigenic human mammary stromal fibroblasts interspersed in mouse adipose tissue; (ii) an epithelial outgrowth comprised of human breast epithelial cells; and (iii) human breast ducts. In a specific embodiment, the human ducts are capable of making milk when the mouse is pregnant.

In another embodiment, the invention provides a mouse having at least one functional humanized mammary gland, wherein the humanized mammary gland comprises: (i) a humanized mammary fat pad comprised of nontumorigenic human mammary stromal fibroblasts interspersed in mouse adipose tissue; (ii) an epithelial outgrowth comprised of human breast epithelial cells. In one embodiment, the epithelial outgrowth comprises human breast ducts. In a further specific embodiment, the human breast ducts produce milk when the mouse is pregnant. In other embodiments of the methods provided herein, the mice are immunocompromised.

The invention also provides mice having two or more humanized mammary gland, where at least two of the humanized mammary glands are generated by a different method. For example, one humanized mammary gland may be generated using genetically modified epithelial cells while a second humanized mammary gland may be generated using epithelial cells that are not genetically modified; or a first humanized mammary gland may be generated using epithelial cells isolated from one patient, while a second humanized mammary gland is be generated using epithelial cells isolated from a second patient.

The humanized mammary fat pad used to generate the humanized mammary gland, can be made according to any of the methods and variations described herein. Accordingly, the mice of the present invention which have a humanized mammary gland can have any or all the variations described for a humanized mammary fat pad.

The human mammary epithelial cells of the methods and mice of the present invention can be derived from preparations of mammary epithelial cell (MEC) organoids. These organoid preparations are clusters of human myoepithelial and luminal epithelial cells, each containing 100 to 10,000 human cells that no longer contain or represent any preexisting structure from the human breast (Friedrichs et al. Cancer Res. 1995; 55:901-906). In one embodiment, 15-30 organoids are pooled and dissociated using a 23 gauge needle and syringue. Alternatively, human mammary epithelial cells may be purchased from commercial sources, such as from Clonetics (Cambrex). Methods for isolating human mammary epithelial stem cells have been described in the art, such as in Smith et al., Microsc Res Tech. 2001; 52(2):190-203; Welm et al. Cell Prolif. 2003; 36 Suppl 1:17-32; Dontu et al. Genes Dev. 2003, 17(10):1253-70; and Trosko et al. Oncol Res. 2003;13(6-10):353-7, the contents of which are hereby incorporated by reference.

From about 1 to about 100,000 human mammary epithelial cells and/or human breast stem cells are injected into a humanized mammary fat pad to generate a humanized mammary gland. One skilled in the art can determine the optimal number for a given application. For example, a composition comprising mostly mammary epithelial cells would contain more cells than one containing mostly breast stem cells. In one embodiment, the composition additionally comprises phosphate buffer saline, cell culture media, matrigel, or collagen, or a combination thereof. In one embodiment, the composition comprises matrigel and collagen from about a 1:1 to a 3:1 ratio.

In preferred embodiments, the human mammary epithelial cells comprise myoepithelial cells and luminal epithelial cells. In a specific embodiment, both myoepithelial cells and luminal epithelial cells are introduced into the humanized mammary fat pad, such as by injection, to generate a humanized mammary gland, while in another specific embodiment myoepithelial cells, luminal epithelial cells and breast stem cells are introduced.

In the methods for generating a humanized mammary gland from a humanized mammary fat pad described herein, the introduction of a composition comprising human mammary epithelial cells into the humanized mammary fat pad is done after the nontumorigenic human stromal fibroblasts of the fat pad have had sufficient time to divide and invade into the adipose tissue of the mammary fat pad. For example, this might be from about 1 to about 60 days, from about 7 to about 21 days, or about two weeks after the nontumorigenic human stromal fibroblasts have been introduced into the cleared mouse mammary fat pad. I one embodiment, the introduction of the composition comprising human mammary epithelial cells and/or human breast stem cells into the humanized mammary fat pad is done at about puberty or after puberty, and hormones are administered to the mouse. In one embodiment, the introduction of a composition comprising human mammary epithelial cells into the humanized mammary fat pad is done by injecting the composition into the same general area as the injection of the stromal fibroblasts.

In a one embodiment, human mammary epithelial cells are mixed with human breast fibroblasts and the resulting composition is then introduced, such as by injection, into the humanized mammary fat pad. Human breast fibroblasts can also be isolated from MEC organoids (Gudjonsson et al., Genes Dev. 2002; 16(6):693-706). In specific embodiments, from about 0 to about 500,000 human breast fibroblasts are coinjected with the human mammary epithelial cells and/or with the human breast stem cells into a humanized mammary fat pad to generate a humanized mammary gland. An exact number may be determined empirically by one skilled in the art. Thus in one embodiment, the humanized mammary glands of the mice described herein are comprised of human breast fibroblasts and human epithelial cells.

In one embodiment, at least one of the human mammary epithelial cells, one of the human mammary stromal fibroblasts, one of the human breast fibroblasts, one of the human breast stem cells, one of the non-human xenogenic stromal fibroblast, or a combination thereof, used in the methods and mice of the present invention are genetically modified, such as by introduction of a transgene or through a mutation that reduces or increases the activity of an endogenous gene, such as a growth factor gene, and oncogene or a tumor suppressor. In one embodiment, the genetic modification results in the altered function of an endogenous oncogene or tumor suppressor gene. In another embodiment, the genetic modification increases the propensity for tumor formation. In another embodiment, the genetically modified comprises an expression construct having a constitutive or an inducible promoter. Transgenes can be introduced into epithelial cells or fibroblasts, for example, using retroviruses or lentiviruses. Transgenes may include, but are not limited to growth factors, receptors, oncogenes, tumor suppressors, cytokines, chemokines, signaling molecules, cell cycle genes such as cyclins or cyclin dependent kinases or phosphatases, and growth factor receptors. In specific embodiments, the transgene may be GFP, prolactin, erbB2 (her2), cyclin D1, EGF receptor, estrogen receptor, sip53 or siBRCA1. In some embodiments, transgenes encode functional polypeptides, mutant polypeptides, dominant negative polypeptides, or encode inhibitory RNAs, such as double stranded or hairpin RNAs. In specific embodiments, genetic modifications the human mammary fibroblasts included a plurality of modifications, such as a cell which contains a knockout mutation and which expresses one or more transgenes.

In another embodiment, the human mammary epithelial cells of the present invention are tumorigenic. In another embodiment, the human breast stem cells can divide and differentiate to generate human mammary epithelial cells which are tumorigenic. These epithelial cells might be derived from human breast tumors, premalignant tissue, tumor cell-lines, benign breast lesions, or by genetic modification of normal epithelial cells. In another embodiment, the human mammary epithelial cells or the human breast stem cells are isolated from women at risk of developing breast cancer, such as those with genetic predispositions. In a particular embodiment, these women at risk for developing breast cancer have not yet been diagnosed with breast tumors, breast cancers or other malignant breast growths.

In another embodiment of the methods and mice of the invention, a mammary fat pad from a mouse can be transplanted into a second mouse, and the transplanted mammary pad may be used to reconstitute a humanized mammary gland. The donor mouse may be of the same or a different genetic background as the recipient mouse. For example, the donor mouse might be an HGF transgenic mouse overexpressing HGF while the recipient is a NOD/SCID mouse.

Human mammary epithelial cells that are injected into the humanized mammary fat pad proliferate and undergo morphogenesis to generate an epithelial outgrowth. In one embodiment, the epithelial outgrowth has a ductal, lobular or acinar morphology. In a further embodiment, the epithelial outgrowth comprises human breast ducts, acini, or both. In another embodiment, the epithelial outgrowth produces milk when the mouse has given birth. In another embodiment, the epithelial outgrowths comprise human acini which produce milk.

To induce full differentiation and morphogenesis of the epithelial outgrowth, the mouse can be made pregnant. Therefore, one embodiment of the present invention provides a pregnant mouse having at least one humanized mammary gland. Alternatively, hormones can be administered to the mouse to mimic a pregnant or a specific hormonal state. In one specific embodiment, the human breast ducts produce milk when the mouse is pregnant.

In one embodiment, the epithelial outgrowth further comprises ductal hyperplasia, carcinoma in situ or invasive ductal carcinoma, or a combination thereof. In a specific embodiment, the epithelial outgrowth comprises hyperplastic or neoplastic outgrowths, such as tumors or carcinomas. Mice having humanized mammary glands with hyperplastic outgrowths or tumors are particularly useful to screen for agents, such as drugs, that inhibit the growth or development of the hyperplastic outgrowths.

Another aspect of the invention provides a mouse that is generated by any of the methods described herein. In one embodiment the mouse has at least one humanized mammary fat pad, while in another embodiment the mouse has at least one humanized mammary gland.

### V. METHODS OF USING RODENTS HAVING HUMANIZED MAMMARY GLANDS

The invention provides methods of using rodents, such as mice, having at least one humanized mammary gland. These methods may employ any of the mouse variants described in the present invention, or mice made by any of the methods disclosed in the instant application.

The invention provides a method of identifying an agent that affects the growth of human breast epithelial cells, comprising (a) contacting the agent with a humanized mammary gland of a mouse, wherein the humanized mammary gland is comprised of (i) a humanized mammary fat pad comprised of human stromal fibroblast cells interspersed in mouse adipose tissue; and (ii) an epithelial outgrowth comprised of human breast epithelial cells; and (iii) human breast ducts; and (b) detecting an effect of the agent on the growth of the breast epithelial cells; and (c) selecting the agent which increases or decreases the growth of the breast epithelial cells. In one embodiment, the human breast ducts produce milk when the mouse is pregnant.

As used herein, the term “agent” includes both compositions, environmental factor, and methods of treatment. An agent can a composition, such as a drug, a small molecule, a polypeptide including modified peptides such as by pegylation, a nucleic acid including single and double stranded DNA and RNA, a virus, a peptide, a hormone such as estrogen or leutenizing hormone, a growth factor or a cell. The agent can also be a method of treatment, such as a surgical procedure or laser treatment. The agent can be an environmental factor, such as humidity, temperature, noise, diet, exercise, or sexual activity. The agent may act directly on the humanized mammary gland, such as a laser treatment of the mammary gland, or the agent may act primarily at another organ or part of the mouse, such as an occlusion of renal arteries to cause kidney failure.

An agent can be any chemical (element, molecule, compound), made synthetically or made by a recombinant technique or isolated from a natural source. For example, agents can be peptides, polypeptides, antibodies or antibody fragments, peptoids, sugars, hormones, or nucleic acid molecules (such as antisense or RNAi nucleic acid molecules). In addition, agents can be small organic molecules or molecules of greater complexity made by, for example, combinatorial chemistry. These libraries can comprise, for example, alcohols, alkyl halides, amines, amides, esters, aldehydes, ethers and other classes of organic compounds. Agents can also be natural or genetically engineered products isolated from lysates or growth media of cells—bacterial, animal or plant.

In one embodiment of the methods described herein, the agent is an RNAi construct. As used herein, the term “RNAi construct” includes small interfering RNAs (siRNAs), hairpin RNAs, and other RNA species which can be cleaved in vivo to form siRNAs. The term RNAi construct also includes expression vectors (also referred to as RNAi expression vectors) that give rise to transcripts which form dsRNAs or hairpin RNAs in cells, and/or transcripts which can produce siRNAs in vivo. As used herein, “dsRNA” refers to double stranded RNA. In some embodiments, dsRNA further contains DNA nucleotides at the 5′ and/or 3′ ends of either or both RNA strands.

In one embodiment of the methods described herein, RNA interference (RNAi) effects knockdown of a protein, such as a protein suspected of modulating tumor formation. RNAi constructs comprise double stranded RNA that can specifically block expression of a target protein. RNAi provides a useful method of inhibiting gene expression in vitro or in vivo. RNAi constructs can comprise either long stretches of dsRNA identical or substantially identical to the target nucleic acid sequence or short stretches of dsRNA identical to substantially identical to only a region of the target nucleic acid sequence.

The subject RNAi constructs can be “small interfering RNAs” or “siRNAs.” These nucleic acids are around 19-30 nucleotides in length, and even more preferably 21-23 nucleotides in length. The RNAi construct can also be in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell.

The agents used in the methods described herein may be polypeptides. The agent may be an antibody. The various antibody molecules and fragments may derive from any of the commonly known immunoglobulin classes, including but not limited to, IgA, secretory IgA, IgE, IgG and IgM. IgG subclasses are also well known to those in the art and include, but are not limited to, human IgG1, IgG2, IgG3 and IgG4.

The agents used in the methods described herein may include antineoplastic agents such as allopurinol sodium, dolasetron mesylate, pamidronate disodium, etidronate, fluconazole, epoetin alfa, levamisole HCL, amifostine, granisetron HCL, leucovorin calcium, sargramostim, dronabinol, mesna, filgrastim, pilocarpine HCL, octreotide acetate, dexrazoxane, ondansetron HCL, ondansetron, busulfan, carboplatin, cisplatin, thiotepa, melphalan HCL, melphalan, cyclophosphamide, ifosfamide, chlorambucil, mechlorethamine HCL, carmustine, lomustine, polifeprosan 20 with carmustine implant, streptozocin, doxorubicin HCL, bleomycin sulfate, daunirubicin HCL, dactinomycin, daunorucbicin citrate, idarubicin HCL, plimycin, mitomycin, pentostatin, mitoxantrone, valrubicin, cytarabine, fludarabine phosphate, floxuridine, cladribine, methotrexate, mercaptipurine, thioguanine, capecitabine, methyltestosterone, nilutamide, testolactone, bicalutamide, flutamide, anastrozole, toremifene citrate, estramustine phosphate sodium, ethinyl estradiol, estradiol, esterified estrogens, conjugated estrogens, leuprolide acetate, goserelin acetate, medroxyprogesterone acetate, megestrol acetate, levamisole HCL, aldesleukin, irinotecan HCL, dacarbazine, asparaginase, etoposide phosphate, gemcitabine HCL, altretamine, topotecan HCL, hydroxyurea, interferon alfa-2b, mitotane, procarbazine HCL, vinorelbine tartrate, E. coli L-asparaginase, Erwinia L-asparaginase, vincristine sulfate, denileukin diftitox, aldesleukin, rituximab, interferon alfa-2a, paclitaxel, docetaxel, BCG live (intravesical), vinblastine sulfate, etoposide, tretinoin, teniposide, porfimer sodium, fluorouracil, betamethasone sodium phosphate and betamethasone acetate, letrozole, etoposide citrororum factor, folinic acid, calcium leucouorin, 5-fluorouricil, adriamycin, cytoxan, and diamino dichloro platinum.

In one embodiment of the methods described herein, the agent or drug is an agent, such as tamoxifen, that can block the production of natural hormones like estrogen, or may comprise aromatase inhibitors which prevent the synthesis of estradiol.

The invention also provides a method of identifying a drug that inhibits abnormal growth of human breast epithelial cells, comprising (a) contacting a candidate drug to be assessed with a humanized mammary gland of a mouse, wherein the humanized mammary gland is comprised of (i) a humanized mammary fat pad comprised of human stromal fibroblast cells interspersed in mouse adipose tissue; (ii) an epithelial outgrowth comprised of human breast epithelial cells; and (iii) human breast ducts; and (b) determining if the candidate drug inhibits abnormal growth of the human breast epithelial cells; and (c) selecting the candidate drug that inhibits abnormal growth of the human breast epithelial cells. In one embodiment, the human breast ducts produce milk. If the candidate drug inhibits abnormal growth, it is a drug that may further be tested as a therapeutic in treating or preventing the development of breast tumors or breast cancers in humans. When a drug is administered to a mouse which has at least one humanized mammary gland to ascertain if the drug inhibits the abnormal growth of human mammary epithelial cells or the formation of tumors, it is advantageous for the untreated control mouse to develop tumors.

Drugs or agents may be administered to the mice of the present invention through oral, rectal, intravaginal, topical, nasal, opthalmic, or parenteral administration. Furthermore, the drugs or agents may be administered locally or systemically, and may be adminstered as part of a dosing regimen requiring single or repeated administrations. As used herein, “parenteral” includes but is not limited to subcutaneous, intravenous, intramuscular, or intrastemal injections or infusion techniques. As used herein, “administering” may be effected or performed using any of the methods known to one skilled in the art. In one embodiment, the agent or drug is introduced into the food or drink of the mouse for oral consumption. In another embodiment, the drug or agent is administered by transdermal injection or by injection into the tail vein.

Determining if a drug inhibits abnormal growth, differentiation or morphogenesis of the human mammary epithelial cells, or if the human mammary epithelial cells are more or less sensitive to the drug, can be done using methods known to those skilled in the art. In one embodiment, the amount of programmed cell death of human mammary epithelial cells in response to the agent or drug is measured. In another embodiment, the level of angiogenesis in response to the agent or a drug may be used as an assay. In a specific embodiment, the number of hyperplastic growths such as ductal hyperplasias, carcinomas in situ or invasive ductal carcinomas in the humanized mammary glands from mice contacted with and without the drug, are compared. A decrease in the extent to which hyperplasia occurs indicates that the drug inhibits abnormal growth of human mammary epithelial cells. In another embodiment, the distribution of hyperplastic events in a humanized mammary gland, such as ductal hyperplasias, carcinomas in situ and invasive ductal carcinomas which reflect the progression of cancer in humans, is compared in mice to whom a candidate drug has been administered and an appropriate control, such as mice who have not received the drug, to determine if a drug reduces the incidence of a particular type of hyperplasia. This embodiment is useful to determine if a drug preferentially blocks one step of cancer progression.

In other embodiments, the size of the abnormal growth or tumor can be determined in mice administered the drug and in control mice. In other embodiments of the methods described herein, multiple agents are tested for their effects on the growth, differentiation or morphogenesis of the human breast epithelial cells. One skilled in the art may chose the appropriate control to use in the methods described herein for identifying an agent or a drug. In one embodiment, the control animal comprises an animal identical or substantially identical to the experimental animal but to which no drug or agent is administered. In another embodiment, the control animal may receive a lower dosage of the drug or agent than the experimental animal. In another embodiment, the agent or drug is administered locally to one humanized mammary gland in a mice having two humanized mammary gland, such that the second mammary gland may serve as a control.

In one aspect, the invention provides methods of determining if a drug is specific for breast cancer. For example, candidate anti-breast cancer drugs can first be screened for their effects on other assay systems such as (i) breast cancer cell lines in culture; (ii) primary cancer cells in culture; or (iii) human breast cancer lines transplanted into mice. Drugs that show therapeutic promise may then be tested using any of the mouse variants described in the invention that have a humanized mammary gland.

Furthermore, the invention provides methods to test for the specificity of a drug for treating a given form of breast abnormality, such as cancer. In one embodiment, the mice used in the assays described herein have breast cancers reconstituted from patients having a specific form of breast cancer. For instance, in one embodiment, breast tumors are reconstituted from patients carrying mutations or altered gene expression of erbB2/neu, BRCA1, BRCA2 or p53 genes to assay for the specificity of a drug for a particular type of breast cancer. One skilled in the art may chose any type of breast cancer or pre-malignant tissue.

The mice of the present invention may also be used for assess the effects of a candidate therapeutic in any breast abnormality. In fact, the utility of the mice is not limited to nay particular type of breast disorder. In one embodiment, the breast disorder is selected from the group consisting of fibroadenoma, papilloma, adenosis, epitheliosis, blunt duct adenosis, sclerosing adenosis, nodular adenosis, and florid adenosis, microglandular adenosis, tubular adenosis, apocrine adenosis, and myoepithelial (adenomyoepithelial) adenosis.

The invention also provides variations of the methods for identifying agents and drugs described herein where the drug or agent is administered before the mice develop functional humanized mammary glands. In one embodiment, the agent is administered before the humanized mammary fat pad is generated, before the human breast epithelial cells or the human breast stem cells are introduced into the fat pad, or before the human breast epithelial cells or the human breast stem cells have divided and form functional outgrowths.

The invention further provides methods of studying agents which affect the development of human breast from human breast stem cells. In one embodiment, human breast stem cells are introduced into humanized mammary fat pads to study their development into human breast. Factors or drugs may be screened for their effects on the proliferation and differentiation of these cells into ductal, acinar or epithelial cell types.

In the methods described herein for generally assessing the effects of a drug or agent on the development or morphogenesis of a humanized mammary gland, any invasive or noninvasive techniques known to one skilled in the art may be employed. Non-invasive techniques include NMR, CAT scans, fluoroscopy, roentgenography, radionuclide scanning, ultrasonography, electrocardiography, electroencephalography, evoked potentials, etc. Invasive techniques include biopsy, autopsy, laparotomy, laparoscopy, intermittent intravenous blood sampling, or intravenous catheterization, etc. Convenient placement of various devices, e.g., catheters, electrodes, etc., may be performed for continuous monitoring.

In some of embodiments of the methods and mice described herein, the human breast ducts make milk or are capable of making milk. In a specific embodiment, the human breast ducts can make milk when the mouse is pregnant, when the mouse is treated with hormones, or when the humanized mammary glands are stimulated physically. It is readily apparent to one skilled in the art that a mouse having a functional human breast duct which makes milk, and therefore is said to be a functional human breast duct, can be made not to secrete milk if, for example, the genes encoding the milk proteins were deleted or otherwise genetically altered in the human mammary epithelial cells or in the human breast stem cells. Likewise, treatment of pregnant mice with drugs or hormones may prevent an otherwise pregnant mice having functional breast ducts unable to make milk under those conditions, but which would otherwise be able to make milk in the absence of the drug or hormonal treatment. Accordingly, mice which have human breast ducts capable of making milk, but which are genetically modified or otherwise treated with an agent to prevent the production of milk, are still considered to be functional human breast ducts, and therefore such mice are within the scope of the invention.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Methods in Mammary Gland Biology and Breast Cancer Research (Margot M. ed Kluwer Academic Publishers, 2000); Epithelial Cell Culture (Ann Harris, ed., Cambridge University Press, 1996). The teachings of these references are hereby incorporated in their entirety.

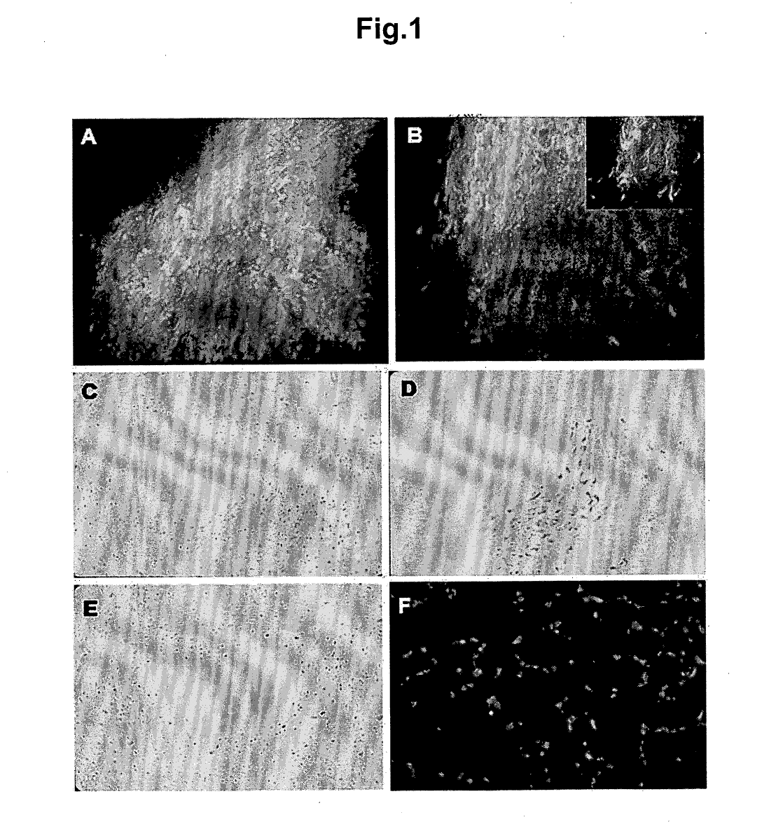

Applicants incorporate by reference FIGS. 1-5, in color, from copending U.S. Application No. 60/520,993, filed Nov. 18, 2003. Furthermore, applicants incorporate by reference the entire teachings, including the color figures, of Kuperwasser et al. Reconstruction of functionally normal and malignant human breast tissues in mice. Proc Natl Acad Sci USA. 2004;101(14):4966-71.

### INCORPORATION BY REFERENCE

The contents of any patents, patent applications, patent publications, or scientific articles referenced anywhere in this application are herein incorporated by reference in their entirety.

### EXEMPLIFICATION

The invention now being generally described, it will be more readily understood by reference to the following examples, which are included merely for purposes of illustration of certain aspects and embodiments of the present invention, and are not intended to limit the invention.

Experimental Procedures

The following experimental procedures were followed in the experimental examples described herein.

Plasmids, Tissue Culture, and Cells

All human breast tissue procurement for these experiments was obtained in compliance with the laws and institutional guidelines, as approved by the institutional IRB committee from Brigham & Women's Hospital and the COUHES committee at MIT. The tissues were obtained from discarded material from patients between the ages of 29-37 years undergoing elective reduction mammoplasty.

Primary human breast fibroblasts and organoids were isolated from reduction mammoplasty tissue as previously described (Pechoux et al. Developmental Biology. 1999; 206:88-99; Pechoux et al. Developmental Biology. 1999;206:88-99). Briefly, breast tissue was chopped into 1 mm cubes and dissociated for 12 hours in a solution of collagenase (3 mg/ml) and hyaluronidase (600 ug/ml). Fibroblasts were separated from organoid-epithelium by differential centrifugation. Fibroblast cells were grown in DMEM supplemented with 10% calf serum and cultured for less than 14 days to expand the cells. Organoids were frozen in cell freeze medium until surgery.

To generate the breast fibroblasts utilized for “humanization” of mammary fat pads termed RMF/EG, primary breast fibroblasts were infected with pLUC-hTERT. Polyclonal populations of fibroblasts were naturally selected through serial passages until a pure GFP positive population of cells emerged as examine by FACs analysis.

These fibroblasts were subsequently subjected to retroviral infections of HGF or TGFβ with drug selection used to purify polyclonal-infected populations after each infection as previously described (Elenbaas et al. Genes Dev. 2001;15:50-65). Drug selection of infected fibroblasts was performed with 700 μg/ml zeocin (zeo).

Retroviral constructs were generated to encode for the catalytic subunit of human telomerase (hTERT) and the green fluorescent protein (pLUC-hTERT). Retroviral constructs for hepatocyte growth factor (HGF) and transforming growth factor beta (TGFβ) were generated by cloning the full length cDNA of HGF (kindly provided by G. Vande Woude) or TGFβ (kindly provided by R. Derynck) into the pBABE-zeo vector system (Clonetech).

Animals and Surgery

A colony of immunocompromised NOD/SCID mice was maintained in-house under aseptic sterile conditions. Mice were administered autoclaved food and water ad-libum. Surgeries were performed under sterile conditions and animals received antibiotics in the drinking water up to 2 weeks following all surgical procedures.

Three week-old female mice were anesthetized and the mammary epithelium was removed from the #4 inguinal mammary glands of NOD/SCID female mice. Two weeks later, 2.5×10⁵ unirradiated RMF/EG fibroblasts and 2.5×10⁵ irradiated (4 Gy) fibroblasts were injected into the cleared fat pads 24 hours after irradiation. Two weeks after the introduction of the human stromal cells, human breast epithelial organoids and primary fibroblasts were injected into the humanized site. For this, organoid preparations were thawed and 20-25 organoids were mixed with 2.5×10⁵ primary fibroblasts and resuspended in 35 ul of a 3:1 Collagen I (Upstate Biotechnology): Phenol-red free Matrigel (Becton Dickinson) mixture and injected into the humanized mammary glands.

Wholemounts and Immunohistochemistry

For murine wholemount analysis, fresh mammary tissue was flattened and fixed in camoy's solution ( ethanol, glacial acetic acid, chloroform) and subsequently stained with carmine alum (carmine, AlKSO₄). Human breast tissue was analyzed by wholemount using hematoxylin. Briefly, fresh tissue obtained from reduction mammoplasty was sliced into 2 cm×1 mm×1 mm fragments and fixed in 10% neutral buffered formalin. Tissues were defatted through graded organics and alcohols and stained in hematoxylin.

Immunohistochemistry was performed on formalin fixed paraffin embedded tissues. Five micron sections were deparaffinized, rehydrated through graded alcohols and subjected to antigen retrieval for immunohistochemistry. Sections were incubated in mouse monoclonal antibodies against smooth muscle actin (1:50 Novacastra), human-specific vimentin (1:50, Novacastra) estrogen receptor (DAKO) progesterone receptor (DAKO), keratin 19 (Novacastra), PCNA (DAKO), p53 (Santa Cruz), human β-casein (gift from Charles Streuli). Immunocomplexes were visualized by the ABC peroxidase method (Vector Laboratories, Burlingame, Calif.), and sections were counterstained with hematoxylin or methyl green.

Genomic FISH

Tissue sections were assayed for the presence of mouse, and human cells using fluorescence in situ hybridization (FISH) with species-specific genomic probes as previously described (Parrott et al., Mol Cell Endocrinol. 2001;175:29-39). Briefly, 5 μm sections were deparaffinized in xylene, dehydrated in ethanol, treated with NaSCN, RNAase, pepsin and finally HCl, and subsequently dehydrated in 70%, 85%, 100% ethanol series. Sections were denatured in and hybridized with probes against CY3-d-UTP labeled mouse Cot 1 DNA (Gibco), or FITC-12-d-UTP labeled human genomic DNA extracted from lymphocytes. Probe size ranged between 300 and 2000 bp.

DNA Fingerprinting

Genomic DNA was isolated from frozen organoid preparations or five 5 um sections of paraffin embedded normal or tumor outgrowth tissue from xenografted material. Paraffin sections were deparaffinized in xylenes and rehydrated to water in graded ethanols. Tissue sections were scraped from the slides and both these samples, as well as the freshly thawed organoid preparations, were subjected to overnight digestion at 55° C. in 100 mM Tris, 5 mM EDTA, 0.2% SDS, 200 mM NaCl and 100 μg/ml Proteinase K (Sigma) solution. Insoluble debris was discarded and DNA was extracted and precipitated through graded organics.

Samples were subsequently subjected to DNA fingerprinting using Promega GenePrint PowerPlex™ 1.2 System (Promega, Madison, Wis.) as previously described (Mitnik et al. Electrophoresis. 2002;23:719-726). PCR was performed according to the protocols supplied by the manufacturer. Amplification was performed on a GeneAmp 9700 thermal cycler (Applied Biosystems) in 9600 emulation mode with a 96-well block. All solutions were prepared fresh on the day of the experiment.

PCR products were analyzed by a four-color optical excitation and detection system including a 40×, 0.5 numerical aperture microscope objective (Melles Griot) to collect the fluorescence from the DNA sample, a series of dichroic mirrors and band-pass filters (Omega Optical, Brattleboro, Vt.) to separate the fluorescence spectrum, and four photomultiplier tubes—PMTs)—(R928, Hamamatsu) to measure the fluorescence signal. The PMTs were interfaced to a computer data acquisition system with data analysis software. The collected data were processed by in-house software. Allelic ladders standards with 3SD values smaller than 0.5 bp were the only standards considered for analysis.

### EXAMPLE 1

Generation of “Humanized” Mammary Fat Pads

Initially, human MECs were engrafted into cleared mouse mammary fat pads. In doing so, Applicants confirmed earlier reports (Outzen et al. J Natl Cancer Inst. 1975;55:1461-1466) indicating that human epithelial cells are unable to establish themselves in the murine mammary stroma and to participate in normal mammary ductal morphogenesis. For reasons discussed above, the failure of human MECs to properly colonize the mouse mammary fat pad suggested the need to establish human mammary stromal cells in fat pads prior to implantation of the epithelial cells. Accordingly, Applicants developed a line of GFP-labeled, hTERT-immortalized human mammary stromal fibroblasts; these cells had previously been prepared from a reduction mammoplasty. These fibroblasts (hereafter termed RMF/EG) are ostensibly normal and non-tumorigenic. They do not form colonies in soft agar and do not form tumors in immunocompromised mice

When the RMF/EG fibroblasts were injected into the cleared mammary fat pads of unirradiated NOD/SCID mice, the cells survived, but little proliferation or expansion of the introduced cells was observed. Therefore, Applicants sought to facilitate engraftment of the RNF/EG fibroblasts and subsequent proliferation by exploiting observations made by others that irradiated fibroblasts undergo a physiological activation that results in the expression of proteases, matrix proteins and a variety of growth factors (Panizzon et al. Radiat Res. 1988; 116:145-156; Barcellos-Hoff et al. Development. 1989;105:223-235). Accordingly, Applicants irradiated 2.5×10⁵ immortalized human stromal fibroblasts with 4 Gy of radiation and injected these, together with an equal number of non-irradiated fibroblasts into cleared mouse mammary fat pads of NOD/SCID mice. This was done to determine if the irradiated fibroblasts would enable the engraftment and invasion of the co-injected non-irradiated cells in the mouse stromal fat pad.

Mammary fat pads that had been injected with these cell mixtures were examined by carmine wholemount analysis at various times between 2 and 8 weeks post-injection, and no detectable changes were observed. However, examination of the mammary fat pads for GFP expression at 4 and 8 weeks after injection clearly demonstrated sites of engraftment and expansion of the human breast fibroblasts within these fat pads. (FIGS. 1A, 1B). Invasion of individual human fibroblasts into the mouse adipose stroma could be observed at high magnification. In addition, large cords of GFP-labeled fibroblasts could also be detected by histology in some of the fat pads (FIG. 1C).

Immunohistochemistry was initially performed on those chimeric fat pads in which cords of fibroblasts had previously been observed by histology. In these cases, the cords of cells were found to be comprised of proliferating fibroblasts expressing human-specific vimentin (FIGS. 1D,1E) and proliferating cell nuclear antigen (PCNA). However, since the great majority of fat pads did not contain readily apparent cords of engrafted fibroblasts, it was difficult to confirm “humanization” by histology in most cases. Therefore, to address whether the RMF/EG fibroblasts had indeed survived and colonized the mouse mammary cleared fat pads, genomic fluorescence in situ hybridization (FISH) was performed on cleared mammary stromal tissues that did not contain histological evidence of stromal outgrowth at either 4 or 8 weeks after injection. A probe specific for mouse Cot1 DNA or for human genomic DNA (prepared by labeling unfractionated human DNA) was hybridized against sections of the humanized mouse mammary fat pads. Examination of these tissues revealed the existence of the human breast fibroblasts interspersed in murine adipose tissue, indicating that these human cells had indeed survived and integrated within the mouse mammary stroma (FIG. 1F).

### EXAMPLE 2

Development of Human Breast Ducts in Chimeric Mammary Fat Pads

Humanized fat pads were used as tissues into which human MECs were introduced. Accordingly, following the construction of a chimeric mammary fat pad, human MEC organoids were prepared from histologically normal human reduction mammoplasty tissues. Organoids are clusters of human myoepithelial and luminal epithelial cells, each containing 100 to 1000 cells that are no longer organized in structures resembling those in the normal human breast (Friedrichs et al. Cancer Res. 1995;55:901-906). From 20 to 25 organoids were mixed with 2.5×10⁵ primary human breast fibroblasts that had also been recently isolated from reduction mammoplasty tissues. The mixed cell preparations were then introduced into the murine stromal fat pads that had been humanized 2 weeks earlier through the introduction of human stromal fibroblasts.

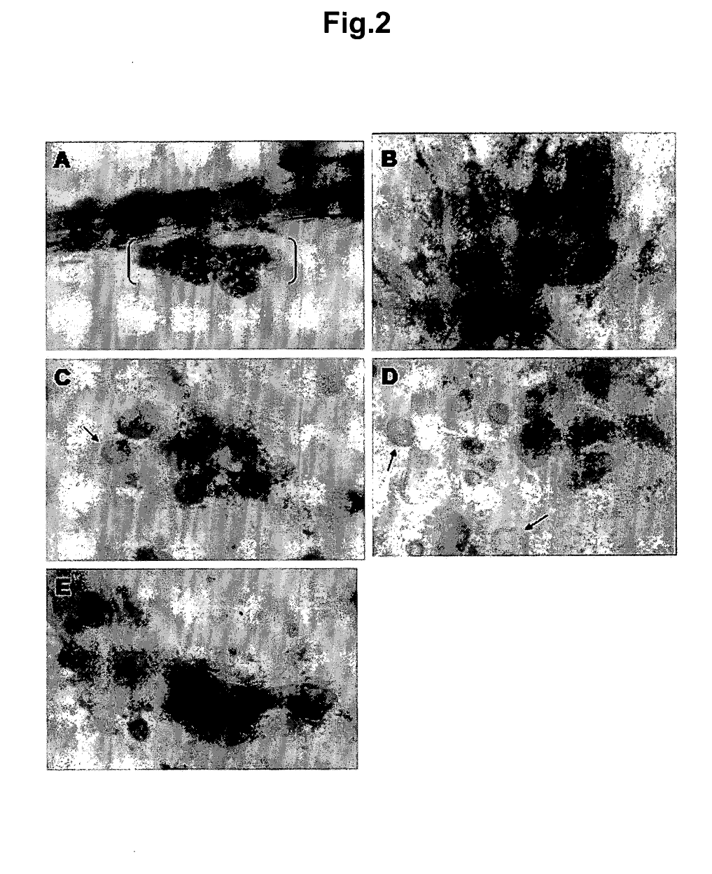

Eight weeks after the introduction of the organoids and admixed fibroblasts, the xenografted mammary glands were removed and subjected to wholemount analysis. Three types of epithelial outgrowths were reproducibly observed among all the xenografts. The relative frequencies of these ductal, lobular and acinar structures varied from one set of donor organoids to another (FIG. 2). When the primary normal breast fibroblasts were not admixed to these organoids prior to engraftment into humanized fat pads, only stunted and deformed looking structures developed.

The most frequently observed outgrowths were of acinar shape, consisting of spherical structures with hollow lumina. Less commonly observed were linear ductal outgrowths with little side-branching (FIG. 2B). As is observed following the standard mouse mammary transplantation technique, ductal development occurred in the absence of additional ectopic estrogen or progesterone hormonal stimulation if the engraftment process was undertaken prior to the end of puberty. Similar to other observations with engrafted murine MECs, the frequency of human ductal elongation decreased significantly when engraftment of the organoids and fibroblasts occurred after puberty in the recipients. In addition to this ductal and acinar development, full ductal-lobular development occurred when mice bearing these human grafts became pregnant (FIG. 2C).

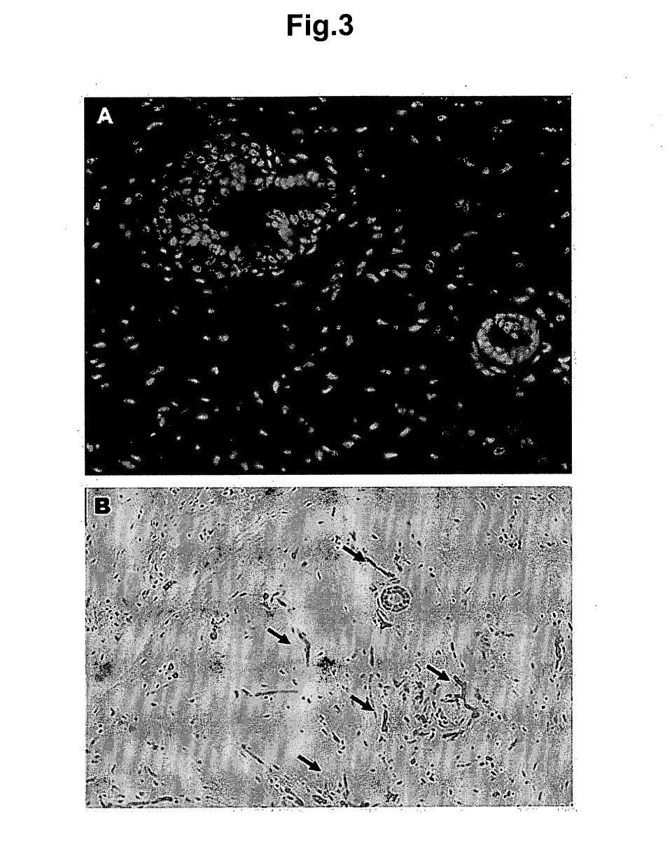

Genomic FISH was performed on paraffin sections of the xenograft outgrowths to determine the origins of the epithelial and stromal cells in the xenografts (FIG. 3A). As anticipated, all of the epithelial structures were of human origin and a significant proportion of the stroma was also of human origin. However, a substantial portion of murine cells could also be found in the stroma. A vascular response was observed within the stroma surrounding the xenografts, as evidenced by the dilation of existing vessels and neo-vascularization that had apparently occurred as a part of normal epithelial development (FIG. 3B).

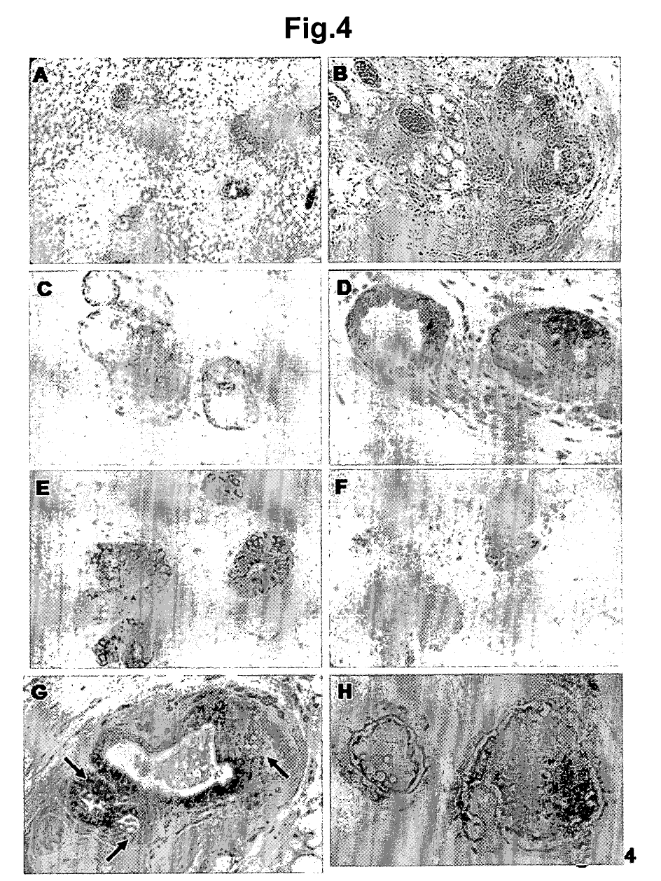

Histological and immunohistochemical analyses were performed on the xenografts in order to examine the morphology and marker expression of the ductal structures (FIG. 4). Normal human mammary ducts are composed of luminal MECs separated from the basement membrane by an intervening layer of myoepithelial cells. To determine if the engrafted human MECs were established in their proper histological positions, myoepithelial cell-specific expression of smooth muscle actin was examined. Indeed, cells located on the basal side of the ducts stained positive for smooth muscle actin and lacked expression of the estrogen receptor, indicating the presence of myoepithelial cells (FIG. 4C). Conversely, luminal cell-specific markers, such as cytokeratin 19, estrogen receptor, and E-cadherin, were expressed in the luminal cells of the ducts (FIGS. 4C and 4E). Finally, sections were examined for the expression of proliferating cell nuclear antigen (PCNA) (FIG. 4F). More than 10% of epithelial cells and 2% of the stromal compartment of the xenografts stained positive for proliferating cell nuclear antigen (PCNA). As normal human breast tissue rarely contains proliferating stromal cells and less than 2% of epithelial cells proliferating at any given time during the menstrual cycle (Shoker et al. Am J Pathol. 1999;155:1811-1815), this indicate that cells in the reconstructed breast tissue were actively proliferating in vivo.

Mice bearing xenografts were mated and allowed to develop until day 18 of pregnancy in order to determine whether full functional differentiation of the human breast tissue had been achieved. Indeed, Applicants observed active lipid synthesis and secretion into the lumina of the human acini (FIG. 4G). Tissue sections were examined for human β-casein expression, and milk protein could be detected both within the luminal MECs lining the acini and in the lumina of the human breast acini (FIG. 4H). Taken together, these various observations indicate that histologically normal, functional human breast tissue can be constructed in mice and that this outcome is dependent on the presence of human stromal cells. Moreover, the mouse mammary stromal fibroblasts that are present in the reconstructed stroma do not appear to impede this process. Furthermore, various stages of human mammary gland development can be recapitulated in vivo, including the production of milk.

### EXAMPLE 3

Hyperplasic and Neoplastic Development of Engrafted Human MECs Due to Stromal Alterations

Applicants speculated that the primary human mammary fibroblasts that were admixed to the human mammary organoids prior to engraftment might in some fashion affect the morphogenetic abilities of the human MECs in these organoids. To address this possibility, Applicants introduced 10 human mammary epithelial organoid preparations isolated from 10 different patients into humanized stromal fat pads either with or without prior addition of primary human mammary stromal fibroblasts. In all cases, histological examination of the mammoplasty specimens had previously revealed no pathological abnormalities.

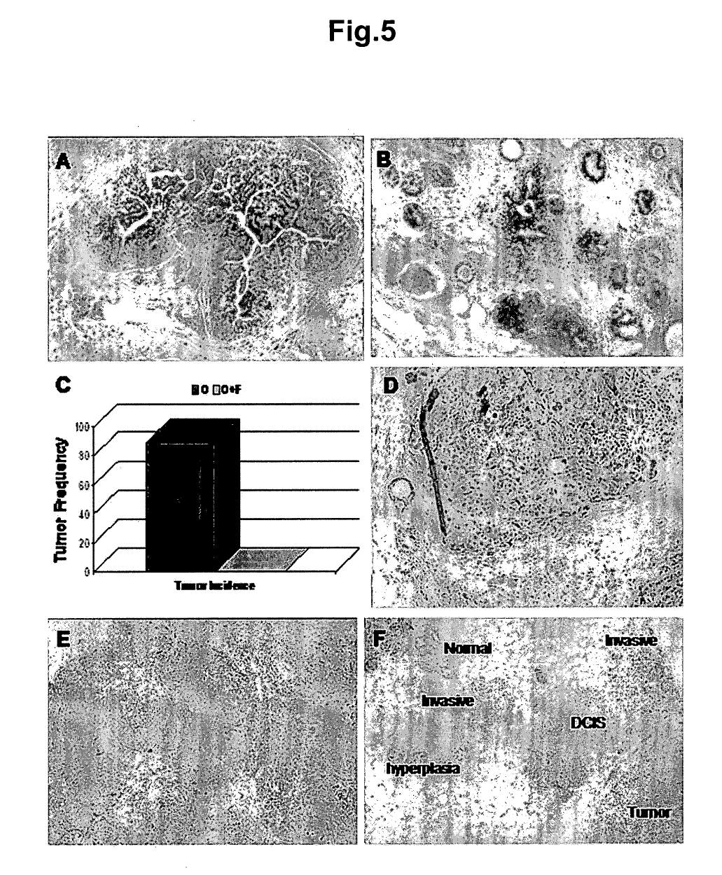

In the nearly 70 co-engraftments with normal stromal fibroblasts, Applicants observed various normal epithelial outgrowths with all 10 organoid preparations. These outgrowths were very similar to those observed in the initial experiment. Different outcomes were seen, however, when primary fibroblasts were not mixed with the organoids prior to implantation,. Implantation of seven of the ten organoid preparations resulted in the formation of normal mammary epithelial structures albeit with somewhat stunted development. In contrast, after implanting ostensibly normal organoids from the remaining three women, Applicants observed abnormal human mammary epithelial structures (FIG. 5A, B). These structures are similar to commonly observed benign human breast proliferations, specifically hyperplastic ductal epithelia. Since Applicants had never observed these hyperplasias in co-engraftments with normal stromal fibroblasts, they concluded that normal primary fibroblasts are capable of suppressing hyperplastic growths that might be generated by preexisting marginally abnormal MECs present in the mammoplasty specimens.

These experiments strongly suggested a critical role for the stroma in controlling mammary epithelial outgrowth. Accordingly, Applicants speculated that further differences between different preparations of organoids if the stromal environment of the engrafted human organoids was perturbed. Thus, stromal cells in normal and neoplastic mammary glands are known to produce certain growth factors that affect the behavior of closely apposed epithelial cells. Consequently, Applicants modified the humanized stroma by forcing the RMF/EG fibroblasts to express either human hepatocyte growth factor (HGF) or human transforming growth factor-β (TGF-β1) prior to using them to humanize the stroma. In mouse mammary glands, overexpression of HGF is known to result in increased branching of the mammary ducts and in a generalized hyperproliferation of the epithelium (Soriano et al. J Cell Sci. 1995;108:413-430). In contrast, TGF-β overexpression inhibits proliferation of MECs (Ewan et al Am J Pathol. 2002;160:2081-2093) but promotes alterations in the stroma, including the conversion of fibroblasts to myofibroblasts, the induction of neoangiogenesis, and other responses associated with wound-healing (Barcellos-Hoff et al. Journal of Mammary Gland Biology and Neoplasia. 1998;3:165-175; Barcellos-Hoff M et al. Cancer Research. 1993;53:3880-3886). Both of these growth factors are known to be synthesized by mammary stromal cells and are found to be overexpressed in the stroma of human breast cancers (Liotta et al. Nature. 2001;411:375-379; Jin et al. Cancer. 1997;79:749-760).

Cleared mammary fat pads were humanized with RMF/EG fibroblasts that overexpress either HGF or TGFβ, or a mixture of both types of fibroblasts. Subsequently, human mammary epithelial organoids prepared from the 10 previously used reduction mammoplasty samples were introduced either directly into these growth factor-enriched fat pads or were mixed with normal primary stromal fibroblasts prior to implantation into the growth factor-enriched fat pads. When mixed with normal primary fibroblasts, Applicants observed normal acini, terminal ductal lobular units (TDLU), and even some ductal outgrowths from the organoids obtained from all 10 patient samples; these structures were very similar to those observed previously when these cells were implanted in humanized stroma that was not engineered to release either of these growth factors.

When the 10 organoid preparations were implanted directly into the growth factor-enriched stromal environment (and thus without prior admixture of normal stromal fibroblasts), 9 of the 10 preparations yielded poorly developed, stunted ductal structures. Surprisingly, this was the case for the 3 samples that had yielded ductal hyperplasias in the previous experiment. While the precise reasons for this disparity are unclear, these experiments indicate the importance of the stromal environment in regulating organoid growth.

One of the 10 reduction mammoplasty specimens behaved quite differently. In this particular case, as described above, cleared fat pads had been humanized with fibroblasts that overexpressed either HGF (12 mammary fat pads), or TGFβ (8 mammary glands), or a mixture of both types of growth factor-overexpressing fibroblasts (16 mammary glands). When primary human breast fibroblasts were mixed with the organoids from this patient prior to engraftment into growth factor-enriched humanized fat pads, normal breast outgrowths were observed in all of the 18 mammary glands (6 HGF mammary glands, 4 TGFβ mammary glands and 8 HGF+TGFβ mixed mammary glands) (FIG. 5C).

However, when the organoids from this patient were introduced directly into the growth factor-enriched, humanized stroma (6 HGF mammary fat pads, 4 TGFβ mammary glands, 8 HGF+TGFβ mammary glands) without admixed primary human stromal fibroblasts, the engrafted MECs developed into growths that closely resembled human ductal carcinomas, including both comedo-type and basal-type carcinomas (FIGS. 5D and 5E). Indeed, carcinomatous growths were seen in all 16 mammary glands (18 less 2 as one TGFβ-humanized animal died prematurely). These tumors were invasive and some were poorly differentiated. Interestingly, in one engrafted mammary gland derived from organoids of this patient, in which the stroma had been humanized with HGF-overexpressing fibroblasts, all of the presumed stages of human breast cancer progression, including hyperplastic ducts, carcinomas in situ, and even invasive carcinomas, could be observed in a single microscope field (FIG. 4F). These tumors were subjected to various immunohistological analyses that confirmed their transformed and invasive phenotypes. There were no significant differences in the histology of the tumors that developed in the fat pads that were humanized with TGFβ alone compared to the HGF or the TGFβ plus HGF fibroblasts. In addition, all of the tumors were confirmed to be of human origin, as ascertained by genomic FISH.

In this experiment, 16 out of 18 mammary gland grafts derived from this preparation of mammoplasty organoids yielded hyperplastic and neoplastic outgrowths. However, when Applicants attempted to reproduce these histopathological abnormalities using organoids prepared from the same patient's mammoplasty sample but from a different region of the original mammoplasty tissue sample, they were unable to do so. We attribute this to inter-regional differences in the biology of various cell populations that preexisted within this breast tissue prior to mammoplasty.

To ensure that the epithelial preparation that gave rise to tumors was indeed from the presumed reduction mammoplasty sample, DNA fingerprinting was performed on tissue sections from the tumor tissues, and organoid preparations from the same patient sample as well as from six other patient samples. In this analysis, eight different chromosomal regions were examined from DNA isolated from the various patient and tissue samples. The microsatellite markers from the tumor tissues and the organoid preparation from the same patient were all identical; and, as anticipated, these differed from the markers associated with the DNAs of the six other patients. Therefore, the tumor phenotype was not the result of contaminating cells from another source.

Taken together, these observations indicate that an altered stromal environment can promote human breast cancer formation by abnormal cells that are present in the normal human breast and elude detection by normal histopathological screening. We speculate that these abnormal cells had already undergone one or several of the steps of tumor progression prior to their excision through mammoplasty. These histological abnormalities, evaluated independently by two breast cancer pathologists, left us with a single clear conclusion: that the mammary gland reconstruction procedure, as described here, can, under certain conditions, allow the formation of many of the steps of human breast cancer progression, including ductal hyperplasia, carcinoma in situ, and invasive ductal carcinoma.

### DISCUSSION OF EXPERIMENTAL EXAMPLES

In attempts to re-create breast tissues in mice, previous studies have attempted to introduce tissue fragments or dissociated MECs into the cleared mouse mammary fat pads of immunocompromised mice (Outzen et al. J Natl Cancer Inst. 1975; 55:1461-1466; Sheffield et al. Int J of Cancer. 1988;41:713-719). These attempts were unsuccessful in re-creating functional, properly differentiated breast tissues, presumably due to the inadequate stromal environment provided by the mouse mammary fat pad.

The success of the irradiated fibroblasts in enabling co-introduced unirradiated fibroblasts to survive and colonize the mammary gland appears to be related to the highly activated microenvironment that is created by these irradiated cells; this microenvironment is characterized by remodeling of the extra cellular matrix (ECM) proteins of the adipose stroma, including increased collagen synthesis and activation of TGFβ ( Barcellos-Hoff et al. Journal of Mammary Gland Biology and Neoplasia. 1998; 3:165-175; Barcellos-Hoff M et al. Cancer Research. 1993; 53:3880-3886; Barcellos-Hoff et al. Radiat Res. 1998; 150:S109-S120). These activities are associated with an activated stromal response and are known to be correlated with fibrosis, invasion, motility and neoangiogenesis in vivo. Due to the high rate of cell death associated with irradiation of fibroblasts, Applicants restricted the irradiation process to only a portion of the fibroblast population prior to engraftment, hoping that the activated irradiated fibroblasts would assist in the survival and engraftment of the non-irradiated fibroblasts within the mammary fat pad of mice—an outcome that was indeed observed.

This humanized stromal environment is hospitable for the engraftment of the human breast epithelial cells in the form of organoids. Proper ductal morphogenesis is dependent on the admixture of primary normal breast fibroblasts to these organoids prior to engraftment into humanized fat pads. This could be due to the fact that in the absence of the co-mingled human fibroblasts, the engrafted human MECs are not initially juxtaposed closely to human stromal cells in the humanized fat pads, depriving them of the human signals they need for ductal elongation and differentiation.

It is worth noting that, as shown here, the systemic hormonal environment of NOD/SCID mice during puberty or pregnancy is sufficient to promote human MEC proliferation and lactogenic differentiation. The use of immunocompromised NOD/SCID mice rather than nude mice in these experiments may account for the fact that additional ectopic estrogen stimulation was not required for MEC proliferation in vivo, as has been reported previously (Parmar et al. Endocrinology. 2002;143:4886-4896). Unlike nude mice, whose reproductive system and thus hormonal environment is compromised (Kopf-Maier et al. J Cancer Res Clin Oncol. 1990;116:229-231), NOD/SCID female mice are fertile with a fully-functional reproductive system. This indicates that the previously observed graft-host inter-species incompatibilities derive from paracrine heterotypic interactions rather than from incompatibilities in the systemic hormonal environment.

We have also demonstrated that histopathologically normal human breast tissues from reduction mammoplasty samples may not behave normally in the absence of a normal stromal environment. We observed this in 3 patient samples that gave rise to hyperplasias in the absence of admixed fibroblasts, and, more strikingly, in one patient sample that gave rise to the various stages of breast cancer progression following implantation without admixed fibroblasts into growth factor-activated, humanized stroma.

Given the rapidity with which these lesions and tumors formed (<8 weeks) and the minimal time they spent outside living tissue (<1 day), Applicants consider it highly unlikely that the human MECs acquired additional genetic alterations after being removed from human breasts and introduced into the immunocompromised mice. We therefore conclude that these human epithelial cells had already undergone one or several premalignant changes prior to their implantation into mice. These abnormal cells were not initially detected by routine histopathological analyses of the donor mammoplasty specimens, either because they were present in only a small proportion of the breast tissue or because their abnormal phenotypes could not be realized in the context of a normal mammary stromal environment.

The notion that occult pre-neoplastic or neoplastic cells exist in breast tissues from young and middle aged women that have not been diagnosed for breast cancer or in tissues obtained from reduction mammoplasties has been evaluated by others (Bhathal et al. Br J Cancer. 1985;51:271-278; Nielsen et al. Br J Cancer. 1987;56:814-819; Alpers et al. Hum Pathol. 1985; 16:796-807; Holst et al. Cancer Res. 2003;63:1596-1601). These histopathological studies have reported that breast tissues from women as early as in their 20s already contain various breast lesions including ductal carcinoma in situ (DCIS), the presumed precursor lesion of ductal carcinomas. Furthermore, evidence suggests that epigenetic alterations of critical growth-regulating genes, such as methylation of the p16INK4A gene promoter, can be detected in morphologically normal breast tissues (Holst et al. Cancer Res. 2003;63:1596-1601). Our results extend these previous reports and demonstrate, moreover, that factors released by stromal cells, together with the presumed preexisting changes in the human mammary epithelial cells, suffice to create outgrowths that are indistinguishable from invasive human breast carcinomas. Given the wide spectrum of epithelial histologies observed in these reconstructed human mammary glands, Applicants conclude that this experimental system provides a unique and novel way to study many of the steps of human breast cancer pathogenesis in vivo, and that, in the future, many of these steps can be mimicked by the genetic modification of the human epithelial or stromal cells prior to their engraftment into mice. Moreover, the construction of analogous models of other human epithelial tissues may also prove useful for understanding a variety of other human neoplasias.

1. A mouse wherein at least one mammary fat pad is a humanized mammary fat pad which comprises nontumorigenic xenogenic mammary stromal fibroblasts interspersed in mouse adipose tissue and supports the morphogenesis of human mammary epithelial cells into human breast ducts.

2. The mouse of claim 1, wherein the human breast ducts produce milk when the mouse is pregnant.

3. A mouse having at least one functional humanized mammary gland, wherein the humanized mammary gland comprises:
(i) a humanized mammary fat pad comprised of nontumorigenic mammary stromal fibroblasts interspersed in mouse adipose tissue; and
(ii) an epithelial outgrowth comprised of human breast epithelial cells.

4. The mouse of claim 3, wherein the epithelial outgrowth comprises human breast ducts.

5. The mouse of claim 4, wherein the human breast ducts produce milk when the mouse is pregnant.

6. The mouse of claim 3, wherein the nontumorigenic human mammary stromal fibroblasts are immortalized nontumorigenic human mammary stromal fibroblasts.

7. The mouse of claim 3, wherein the nontumorigenic human mammary stromal fibroblasts are genetically modified.

8. The method of claim 3, wherein at least one of the nontumorigenic human mammary stromal cells is genetically modified.

9. The mouse of claim 3, wherein the genetic modification results in the altered function of an oncogene or of a tumor suppressor gene.

10. The mouse of claim 3, wherein the genetic modification results in the expression of a polypeptide selected from the group comprising of a catalytic subunit of telomerase, GFP, TGF-β, HGF, FGF-7 and FGF-1, IGF, EGF, CSF-1, PDGF, SDF-1, and heregulin.

11. The mouse of claim 3, wherein the mouse is an immunocompromised mouse.

12. The mouse of claim 11, wherein the mouse is a NOD/SCID mouse, a RAG mouse or a nude mouse.

13. The mouse of claim 3, wherein the epithelial outgrowth additionally contains human breast fibroblasts.

14. The mouse of claim 3, wherein the epithelial outgrowth has a ductal, lobular or acinar morphology.

15. The mouse of claim 3, wherein the human mammary epithelial cells are myoepithelial cells, luminal epithelial cells or a combination thereof.

16. The mouse of claim 3, wherein the human mammary epithelial cells are derived from MEC organoids.

17. The mouse of claim 3, wherein the human mammary epithelial cells are tumorigenic.

18. The mouse of claim 3, wherein the mouse is pregnant.

19. The mouse of claim 3, wherein the human mammary epithelial cells are genetically modified.

20. The mouse of claim 3, wherein the mammary epithelial cells contain a genetic modification that increases the propensity for tumor formation.

21. The mouse of claim 19, wherein the human mammary epithelial cells are genetically modified to express a growth factor, a receptor, an oncogene, a tumor suppressor, or a cell cycle gene.

22. The mouse of claim 19, wherein the human mammary epithelial cells are genetically modified to express GFP, prolactin, erbB2, cyclin D1, EGF receptor, estrogen receptor, sip53 or siBRCA1.

23. The mouse of claim 3, wherein the mouse has two humanized mammary glands.

24. The mouse of claim 23, wherein the humanized mammary glands comprise human breast ducts,

25. The mouse of claim 23, wherein the human breast ducts secrete milk when the mouse if pregnant.

26. The mouse of claim 3, wherein the epithelial outgrowth comprises hyperplastic or neoplastic growths.

27. The mouse of claim 26, wherein the epithelial outgrowth further comprises a ductal hyperplasia, a carcinoma in situ or an invasive ductal carcinoma or a combination thereof.

28. A method of producing a humanized mammary fat pad in a mouse, wherein the humanized mammary fat pad comprises human breast fibroblast cells interspersed in mouse mammary adipose tissue, the method comprising:
(a) generating nontumorigenic human mammary stromal fibroblasts;
(b) treating the nontumorigenic human mammary stromal fibroblasts to induce their proliferation and their invasion into a mouse fat pad, and introducing the nontumorigenic human mammary stromal fibroblasts into a cleared mammary fat pad from the mouse; and
(c) allowing sufficient time for the nontumorigenic human mammary stromal fibroblasts to divide and invade into the cleared mammary fat pad,
thereby producing a humanized mammary fat pad.

29. The method according to claim 28, wherein sufficient time is at least 1 week.

30. The method according to claim 28, wherein the nontumorigenic human mammary stromal fibroblasts are treated to induce their proliferation and their invasion into a mouse fat pad before they are introduced into the cleared mammary fat pad.

31. The method according to claim 28, wherein the nontumorigenic human mammary stromal fibroblasts are treated to induce their proliferation and their invasion into a mouse fat pad after they are introduced into the cleared mammary fat pad.

32. The method according to claim 28, wherein the nontumorigenic human mammary stromal fibroblasts comprise immortalized nontumorigenic human mammary stromal fibroblasts.

33. The method of claim 28, wherein treating the nontumorigenic human mammary stromal fibroblasts to induce their proliferation and invasion into a mouse fat pad comprises irradiating the nontumorigenic human mammary stromal fibroblasts.

34. The method of claim 28, wherein treating the nontumorigenic human mammary stromal fibroblasts to induce their proliferation and invasion into a mouse fat pad comprises introducing one or more transgenes into the fibroblasts.

35. The method of claim 28, wherein at least one of the nontumorigenic human mammary stromal cells is genetically modified.

36. The method of claim 35, wherein the genetic modification results in the altered function of an oncogene or of a tumor suppressor gene.

37. The method of claim 35, wherein the genetic modification results in the expression of a polypeptide selected from the group comprising of a catalytic subunit of telomerase, GFP, TGF-β, HGF, FGF-7 and FGF-1, IGF, EGF, CSF-1, PDGF, SDF-1, and heregulin.

38. The method of claim 28, wherein the at least one of the nontumorigenic human mammary stromal cells is genetically modified to express a double-stranded RNA molecule.

39. The method of claim 38, wherein the double-stranded RNA molecule is expressed constitutively or is expressed under the control of an inducible promoter.

40. The method of claim 28, wherein at least one of the nontumorigenic human mammary stromal cells is genetically modified with an expression construct having a constitutive or an inducible promoter.

41. The method of claim 28, wherein the mouse is an immunocompromised mouse.

42. A mouse comprising at least one humanized mammary fat pad generated according to the method of claim 28.

43. A method of generating a humanized mammary gland, comprising
(a) generating a humanized mammary fat pad;
(b) introducing a composition comprising
(1) human mammary epithelial cells; or
(2) human breast stem cells; or
(3) a combination thereof,
into the humanized mammary fat pad; and
(c) allowing sufficient time and appropriate conditions for an epithelial outgrowth to develop,
thereby forming a humanized mammary gland.

44. The method of claim 43, wherein the composition further comprises human breast fibroblasts.

45. The method of claim 43, wherein the epithelial outgrowth produces milk.

46. The method of claim 43, wherein the epithelial outgrowth comprises hyperplastic or neoplastic growths.

47. The method of claim 43, wherein the human mammary epithelial cells, the human breast stem cells, or both, are genetically modified.

48. The method of claim 43, wherein the human mammary epithelial cells are myoepithelial cells, luminal epithelial cells, or a combination thereof.

49. The method of claim 43, wherein introduction of the composition is performed from about 1 day to about 60 days after generating the humanized mouse pad.

50. A mouse comprising at least one humanized mammary gland generated according to the method of claim 43.

51. A method of identifying an agent that affects the growth of human breast epithelial cells, comprising
(a) contacting a humanized mammary gland of a mouse with the agent, wherein the humanized mammary gland is comprised of
(i) a humanized mammary fat pad comprised of human stromal fibroblast cells interspersed in mouse adipose tissue; and
(ii) an epithelial outgrowth comprised of human breast epithelial cells; and
(iii) human breast ducts; and
(b) detecting an effect of the agent on the growth of the breast epithelial cells; and
(c) selecting the agent which increases or decreases the growth of the breast epithelial cells.

52. A method of identifying a drug that inhibits abnormal growth of human breast epithelial cells, comprising
(a) contacting a humanized mammary gland of a mouse with a candidate drug, wherein the humanized mammary gland is comprised of
(i) a humanized mammary fat pad comprised of human stromal fibroblast cells interspersed in mouse adipose tissue;
(ii) an epithelial outgrowth comprised of human breast epithelial cells; and
(iii) human breast ducts;
(b) determining if the candidate drug inhibits abnormal growth of the human breast epithelial cells; and
(c) selecting the candidate drug that inhibits abnormal growth of the human breast epithelial cells.

53. The method of claim 51, wherein the epithelial outgrowth further comprises a hyperplastic or neoplastic condition selected from the group comprised of ductal hyperplasia, carcinoma in situ or invasive ductal carcinoma.

54. The method of claim 51, wherein the human breast ducts make milk when the mouse is pregnant.

55. The method of claim 51, wherein the human breast epithelial cells have a mutation in an oncogene or in a tumor suppressor.

56. The method of claim 55, wherein the oncogene or tumor suppressor is erbB2, BRCA1, BRCA2 or p53.

57. The method of claim 51, wherein the agent is a drug, a double stranded RNA molecule, a surgical procedure or radiation.

58. The method of claim 43, wherein the composition comprises human mammary epithelial cells.

59. The method of claim 58, wherein the human mammary epithelial cells are nontumorigenic human mammary epithelial cells.

60. The method of claim 58, wherein the human mammary epithelial cells (i) are not genetically engineered, (ii) do not express a recombinant transgene, or (iii) both.

61. The method of claim 51, wherein contacting the agent with a humanized mammary gland of a mouse is affected by administering the agent to the mouse orally or by injection.

62. The method of claim 51, wherein the agent is a polypeptide.

63. The method of claim 62, wherein the polypeptide is an antibody.
